SEQUENCE LISTING (2 sequences)

SEQ ID NO: 1 - DNA, 21 bp, Homo sapiens
ucgagaaucuaaacuaacutt

SEQ ID NO: 2 - DNA, 21 bp, Homo sapiens
aguuaguuuagauucucgatt

(19) United States
(12) Patent Application Publication
(10) Pub. No.: US 2013/0079383 A1
(43) Pub. Date: Mar. 28, 2013

(54) Lipid Compounds Targeting VLA-4

(71) Applicants: John Frederick BOYLAN, Bedminster, NJ (US); Robert A. GOODNOW, Gillette, NJ (US); Wei HE, Livingston, NJ (US); Tai-An LIN, Pequannock, NJ (US); Achyutharao SIDDURI, Livingston, NJ (US)

(72) Inventors: Michael J. BENNETT, Monrovia, CA (US); John Frederick BOYLAN, Bedminster, NJ (US); Robert A. GOODNOW, Gillette, NJ (US); Wei HE, Livingston, NJ (US); Tai-An LIN, Pequannock, NJ (US); Achyutharao SIDDURI, Livingston, NJ (US)

(73) Assignee: ARROWHEAD RESEARCH CORPORATION, Pasadena, CA (US)

(21) Appl. No.: 13/541,903

(22) Filed: Jul. 5, 2012

Related U.S. Application Data
(60) Provisional application No. 61/507,144, filed on Jul. 13, 2011.

(51) Int. Cl. C07C 237/40; A61K 47/18

(52) U.S. Cl. CPC C07C 237/40; A61K 47/186
USPC 514/44 A; 562/448

(57) ABSTRACT

The invention relates to the compounds of formula I:

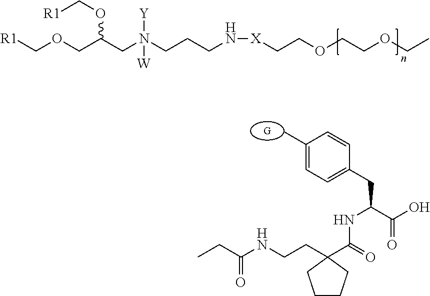

and pharmaceutically acceptable salts and esters thereof, wherein n, G, W, X, Y, and R1 are defined in the detailed description and claims. The compounds of formula I bind to or associate with VLA-4 and can be used in delivery formulations to deliver drugs, nucleic acids, or other therapeutic compounds to tissues or cells expressing VLA-4.

### CROSS-REFERENCE TO RELATED APPLICATIONS

This application claims the benefit of U.S. provisional application No. 61/507,144, filed 13 Jul. 2011.

### FIELD OF THE INVENTION

The present invention relates to lipid compounds that bind to VLA-4 which can be used in delivery formulations to deliver drugs, nucleic acids, or other therapeutic compounds to tissues or cells expressing VLA-4. In particular embodiments, the lipid compounds of the present invention are components of liposomes or lipid nanoparticle compositions that target VLA-4 which are used for delivering nucleic acids such as siRNA to inhibit or prevent the expression of target genes through RNA interference (RNAi).

### BACKGROUND OF THE INVENTION

VLA-4 (integrin alpha-4-beta-1 or Very Late Antigen-4) is an integrin dimer. It is comprised of two subunits consisting of CD49d (alpha) and CD29 (beta). VLA-4 is expressed on leukocyte plasma membranes which bind to VCAM-1 on blood vessels (after activation by cytokines) helping the leukocytes to adhere to vascular endothelium (contributing to atherosclerosis or other inflammatory diseases). Certain cancer cells may also express VLA-4 which bind to VCAM-1 adhering to the endothelium (increasing the risk of metastasis). Thus lipid compounds that bind to VLA-4 may block the interaction with VCAM-1 potentially treating or preventing diseases mediated by this interaction. Alternatively, lipid compounds that bind to VLA-4 may be used in delivery formulations to deliver drugs, nucleic acids, or other therapeutic compounds to tissues or cells expressing VLA-4 for the treatment or prevention of disease.

RNA interference is a well-known process in which the translation of messenger RNA (mRNA) into protein is interfered with by the association or binding of complementary or partially complementary oligonucleotides such as small interfering RNA (siRNA), short hairpin RNA (shRNA), micro RNA (miRNA), or antisense oligonucleotides. siRNAs are double-stranded RNA molecules, usually ranging from 19-25 nucleotides in length that associate with a set of proteins in the cytoplasm known as RISC(RNA-induced silencing complex). RISC ultimately separates the double stranded siRNA allowing one strand to bind or associate with a complementary or partially complementary portion of an mRNA molecule after which the mRNA is destroyed by RISC or otherwise prevented from being translated—consequently suppressing the expression of the encoded protein or gene product.

One of the problems in using nucleic acids such as siRNA in therapeutic applications (especially for systemic administration in humans) has been in delivering the nucleic acids to particular target tissues or cell types and to the cytoplasm of those cells (i.e., where the mRNA is present and translated into protein). Part of the delivery problem is based on the fact that nucleic acids are negatively charged, easily degraded (especially if unmodified), efficiently filtered by the kidney, and cannot be easily transported to the cytoplasm of the cells by themselves. Thus, a significant amount of research has focused on solving the delivery problem with various carriers and formulations including liposomes, micelles, peptides, polymers, conjugates and aptamers (Ling et al. “Advances in Systemic siRNA Delivery” Drugs Future 2009, 34(9):721). Some of the more promising delivery vehicles have involved the use of lipidic systems including lipid nanoparticles. (Wu et al. “Lipidic Systems for In Vivo siRNA Delivery” AAPS J. 2009, 11(4):639-652; Hope et al. “Improved Amino Lipids And Methods For the Delivery of Nucleic Acids” International Patent Application Publication No. WO 2010/042877. However, clinical trials using lipid-based nanoparticles (LNPs) have been somewhat limited by safety concerns, antibody opsonization and phagocytosis, and an inability to deliver nucleic acids, such as siRNA, to organs other than the liver and lung after intravenous administration. Thus, a need remains for further improved carriers and formulations including lipid-based nucleic acid delivery systems capable of safely and efficiently delivering nucleic acids such as siRNA to particular target cells (such as those expressing VLA-4) and to the cytoplasm of such cells, while avoiding or reducing reticuloendothelial clearance and/or opsonization.

### SUMMARY OF THE INVENTION

The invention relates to the compounds of formula I:

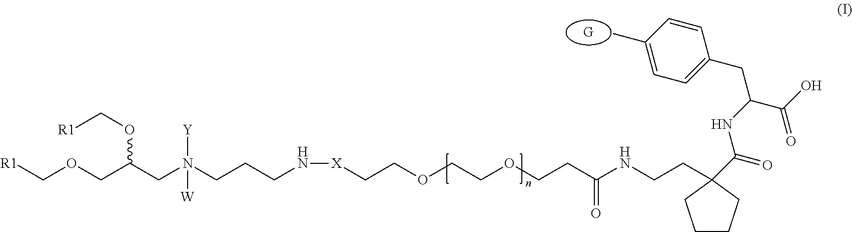

and pharmaceutically acceptable salts and esters thereof, wherein n, G, W, X, Y, and R1 are defined in the detailed description and claims. In addition, the present invention relates to novel compositions and formulations containing compounds of formula I for improved delivery of nucleic acids such as siRNA to the cytoplasm of target cells expressing VLA-4. The present invention also relates to methods of manufacturing and using such compounds and compositions. The compounds of formula I are useful as components of compositions or formulations which improve the delivery of drugs, nucleic acids, or other therapeutic compounds to tissues or cells expressing VLA-4. In particular embodiments, the present invention relates to lipid nanoparticle formulations containing the compounds of formula I which are useful in delivering siRNA to the cytoplasm of target cells expressing VLA-4 to inhibit the expression of certain proteins through RNA interference. In more particular embodiments, the present invention relates to the compounds of formula I and compositions containing such compounds that can effectively deliver siRNA to tumor cells and other cell types expressing VLA-4 for the treatment of cancer or inflammatory diseases. Such compounds and compositions are more efficacious and demonstrate improved knockdown capability compared to similar formulations lacking the compounds of formula I.

### BRIEF DESCRIPTION OF THE DRAWINGS

FIG. 1. Bar graph illustrating KIF11 mRNA knockdown in MV4-11 cells (relative to 18S ribosomal RNA as a control) with various LNP formulations containing KIF11 siRNA (formulation nos. 54F, 54I, 54L, and 54O).

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule. For example, the variables X, Y, and R1 of formula I refer to moieties that are attached to the structure shown in formula I by a covalent bond where indicated.

Unless otherwise indicated, the term “hydrogen” or “hydro” refers to the moiety of a hydrogen atom (—H) and not H₂.

The term “alkyl” refers to an aliphatic straight-chain or branched-chain saturated hydrocarbon moiety having 1 to 25 carbon atoms. In particular embodiments the alkyl has 9 to 25 carbon atoms, preferably 18-20 carbon atoms.

Unless otherwise indicated, the term “a compound of the formula” or “a compound of formula” or “compounds of the formula” or “compounds of formula” means any compound selected from the genus of compounds as defined by the formula (Including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like. Depending on the substitution patterns, the compounds of the present invention may also exist as zwitterions.

The compounds of the present invention can be present in the form of pharmaceutically acceptable salts. The compounds of the present invention can also be present in the form of pharmaceutically acceptable esters (i.e., the methyl and ethyl esters of the acids of formula I to be used as prodrugs). The compounds of the present invention can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “a therapeutically effective amount” of a formulation of siRNA means an amount of an siRNA compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

In detail, the present invention relates to the compounds of formula I:

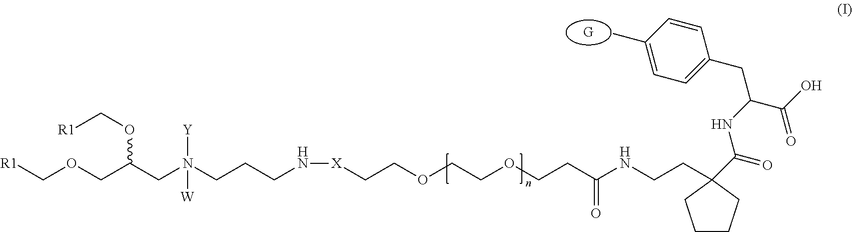

and pharmaceutically acceptable salts and esters thereof, wherein:

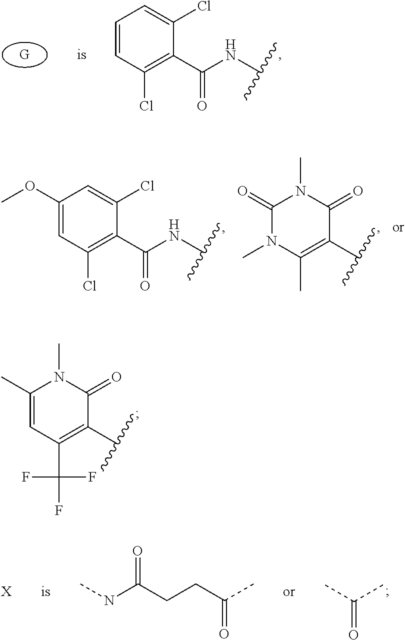

W and Y are hydrogen or methyl, wherein at least one of Y or W is methyl;
R1 is an alkyl having 9 to 25 carbon atoms, and
n is 8 to 25.

In addition, the present invention relates to methods of manufacturing and using the compounds of formula I as well as pharmaceutical compositions containing such compounds. The compounds of formula I are useful in formulating compositions such as nanoparticles to improve the delivery of nucleic acids such as siRNA to the cytoplasm of target cells expressing VLA-4. In particular embodiments, the present invention relates to lipid nanoparticle compositions and formulations containing the compounds of formula I which are useful in delivering siRNA to the cytoplasm of target cells expressing VLA-4 to inhibit the expression of certain target proteins through RNA interference.

In more particular embodiments, the invention relates to the use of the compounds of formula I for formulation into lipid nanoparticle compositions to facilitate the delivery of nucleic acids such as siRNA to tumor cells and other cell types expressing VLA-4. Furthermore, the use of compounds of formula I to synthesize delivery formulations to treat inflammation and proliferative disorders, like cancers, is part of the invention.

In particular embodiments, the present invention is directed to a compound of formula I wherein:

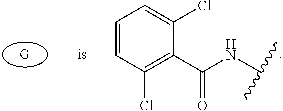 [SMILES: *NC(=O)c1c(Cl)cccc1Cl]

In other embodiments, the present invention is directed to a compound of formula I wherein:

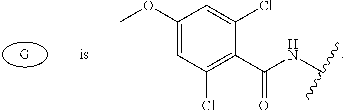 [SMILES: *NC(=O)c1c(Cl)cc(OC)cc1Cl]

In other embodiments, the present invention is directed to a compound of formula I wherein:

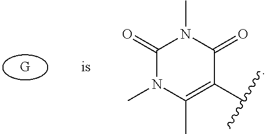 [SMILES: *c1c(C)n(C)c(=O)n(C)c1=O]

In other embodiments, the present invention is directed to a compound of formula I wherein:

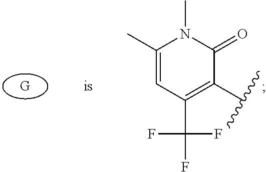 [SMILES: *c1c(C(F)(F)F)cc(C)n(C)c1=O]

In other particular embodiments, the present invention is directed to a compound of formula I wherein X is:

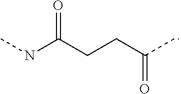

In other embodiments, the present invention is directed to a compound of formula I wherein X is:

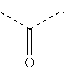

In other embodiments, the present invention is directed to a compound of formula I wherein W is hydrogen and Y is methyl.

In other embodiments, the present invention is directed to a compound of formula I wherein both W and Y are methyl.

In other embodiments, the present invention is directed to a compound of formula I wherein R1 is an alkyl having 9-20 carbon atoms.

In other embodiments, the present invention is directed to a compound of formula I wherein R1 is an alkyl having 16-20 carbon atoms.

In other embodiments, the present invention is directed to a compound of formula I wherein R1 is an alkyl having 18 carbon atoms.

In other embodiments, the present invention is directed to a compound of formula I wherein R1 is an alkyl having 19 carbon atoms.

In other embodiments, the present invention is directed to a compound of formula I wherein R1 is an alkyl having 20 carbon atoms.

In other embodiments, the present invention is directed to a compound of formula I wherein n is 9-13, preferably 12.

In more specific embodiments, the present invention is directed to a compound of formula I selected from the group consisting of:
[2,3-bis(dodecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxo-propyl]amino]propyl]dimethylaminium trifluoroacetate;
[2,3-bis(dodecyloxy)propyl][3-[[4-[[2-[2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1-4-dioxobutyl]amino]propyl]dimethyl-aminium trifluoroacetate;
[[2,3-bis(hexadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxo-propyl]amino]propyl]dimethylaminium trifluoroacetate;
[2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethyl-aminium trifluoroacetate;
(S)-Alpha-[[[1-[2-[[3-[2-[2-[2-[2-[2-[2-[2-[2-[2-[2-[2-[2-[[4-[[3-[[2,3-bis(hexadecyloxy) propyl]methylamino]propyl]amino]-1,4-dioxobutyl]amino]ethoxy]ethoxy]ethoxy]ethoxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino)benzenepropanoic acid;
[2,3-bis(octadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxo-propyl]amino]propyl]dimethylaminium bromide;
[2,3-bis(icosanyloxy)propyl][3-[[3-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxo-propyl]amino]propyl]dimethylaminium bromide;
(S)-Alpha-[[[1-[2-[[3-[2-[2-[2-[2-[2-[2-[2-[2-[[3-[[3-[[2,3-bis(icosanyloxy)propyl]methylamino]propyl]amino]-3-oxopropyl]oxy]ethoxy]ethoxy]ethoxy]ethoxy]ethoxy]ethoxy]ethoxy]ethoxy]-1-oxopropyl]amino]ethyl]cyclopentyl]carbonyl]amino]-4-(2,6-dichloro-benzoylamino)benzenepropanoic acid; and
[2,3-bis(octadecyloxy)propyl][3-[[3-[2-[2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethoxy]-1-oxopropyl]amino]propyl]dimethylaminium trifluoroacetate.

In other particular embodiments, the present invention is directed to lipid nanoparticle compositions comprising:

(1) a compound selected from the group consisting of:
2-(2,2-di((9Z,12Z)-octadeca-9,12-dienyl)-1,3-dioxolan-4-yl)-N,N-dimethylethanamine;
N-(chloromethyl)-2-(2,2-di((9Z,12Z)-octadeca-9,12-dienyl)-1,3-dioxolan-4-yl)-N,N-dimethylethanamonium chloride;
2-(2,2-di((9Z,12Z)-octadeca-9,12-dienyl)-1,3-dioxolan-4-yl)-N,N,N-trimethylethan-amonium iodide; and
2-(2,2-di((9Z,12Z)-octadeca-9,12-dienyl)-1,3-dioxolan-4-yl)-N-ethyl-N,N-dimethylethanamonium iodide;

(2) a compound of formula I (a VLA-4-targeting lipid)

(3) a phospholipid selected from the group consisting of:
1,2-dipalmitoyl-sn-glycero-3-phosphocholine,
1,2-distearoyl-sn-glycero-3-phosphocholine, and
1,2-dioleoyl-sn-glycero-3-phosphocholine;

(4) cholesterol;

(5) hyaluronic acid; and

(6) a polynucleotide (such as siRNA).

### General Synthesis of the Compounds of the Invention

Suitable processes for synthesizing compounds of formula I are provided in the examples. Generally, compounds of formula I can be prepared according to the schemes illustrated below. Unless otherwise indicated, the variables G, W, X, Y, and R1 in the schemes below are defined in the same manner as defined previously for the genus of formula I.

Integrin targeting lipids of compound 1:

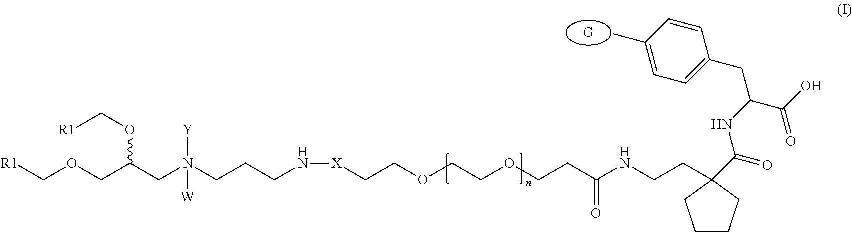

were made in the following general manner. First, intermediate 8 as shown below was made according to a method described in Proc Natl Acad Sci USA: 1996, 93:11454-11459 as shown in Scheme 1.

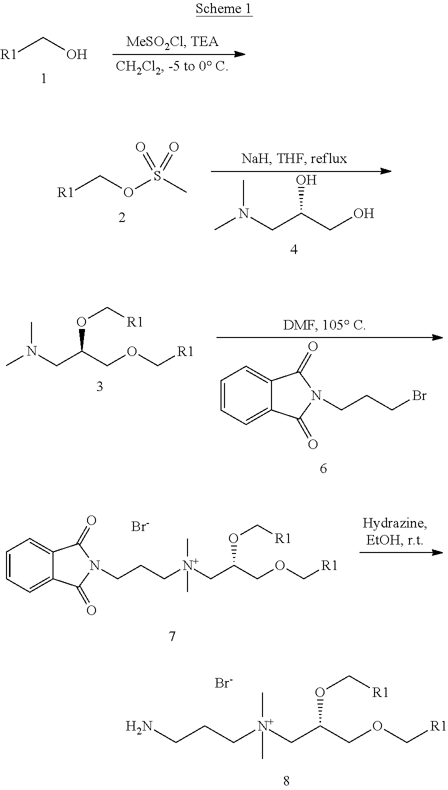

Specifically, primary alcohols wherein R1 is an alkyl having 9 to 25 carbon atoms were treated in an aprotic solvent such as dicholoromethane chilled to −5° C. in presence of a base, preferably triethylamine, with methanesulfonyl chloride, resulting in the formation of sulfonate ester 3. Compound 3 was treated with known compound 4, which had been reacted with sodium hydride in an aprotic solvent such as tetrahydrofuran (THF), resulting in di-ether 5. Di-ether 5 was reacted with commercially available 2-(3-bromo-propyl)-isoindole-1,3-dione 6 in DMF at 105° C. for 3 days. This reaction resulted in quaternary ammonium salt 7, the phthalimide protection group of which was removed by reaction with hydrazine in a lower alkyl alcohol such as ethanol at room temperature (RT) for 3 days. Upon purification, either by chromatography or trituration, amine 8 was obtained.

Next, intermediate 16 can be synthesized in a manner similar to that has been reported (Sidduri A et al. Bioorganic & Medicinal Chemistry Letters, 2002, 12:2475-2478) and as shown in Scheme 2:

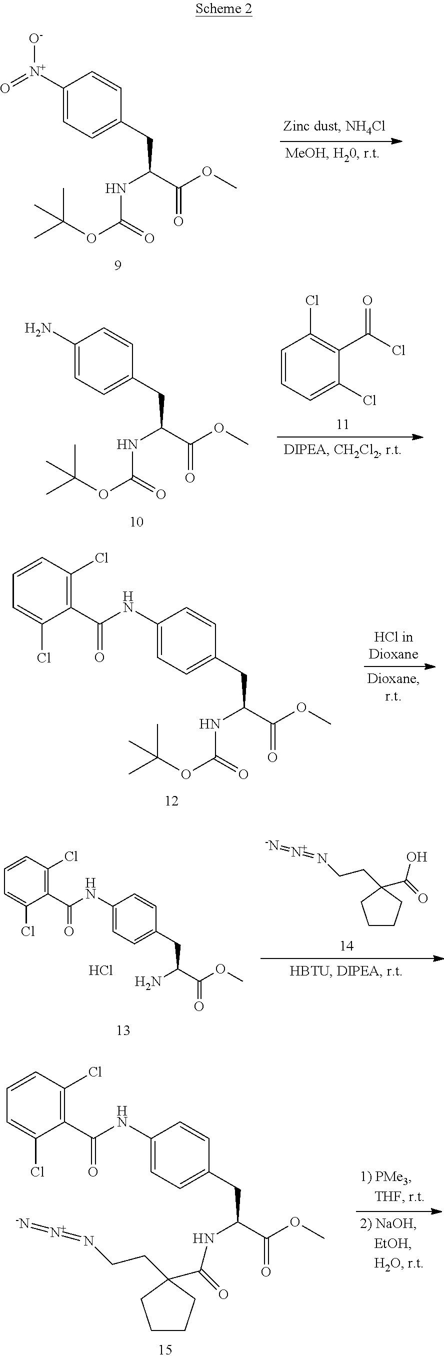

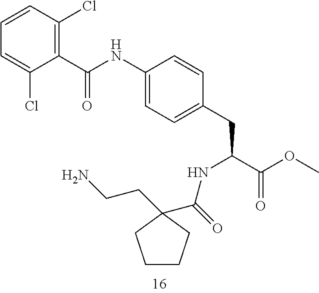 [SMILES: COC(=O)[C@H](Cc1ccc(NC(=O)c2c(Cl)cccc2Cl)cc1)NC(=O)C1(CCN)CCCC1]

Specifically, intermediate 16 was created from commercially available N-Boc-para-nitrophenylalanine 9. The nitro group of commercially available starting material 9 in a methanol solution was reduced with zinc dust in presence of ammonium chloride at RT over the course of several h, resulting in aniline 10. Other methods for nitro reduction are known to those skill in the art. Aniline 10 was acylated with benzoyl halide derivatives such as 2,6-dichlorobenzoyl chloride in aprotic solvent such as dichloromethane in the presence of a base such as di-isopropylethyl amine at RT. In this manner, amide 12 was formed. The t-butylcarbonyl (Boc) amine protecting group was removed according to standard methods known to those skilled in the art, such as by treatment with an HCl solution in dioxane at RT; this resulted in hydrochloride 13. Hydrochloride 13 was treated with amide bond forming conditions (also well known to those skilled in the art) in presence of known 1-(2-azido-ethyl)-cyclopentanecarboxylic acid 14 resulting in the production of di-amide 15. The azide group of intermediate 15 was reduced by treatment with tri-alkyl phosphine in an aprotic solvent such as THF at RT. Further, the methyl ester of intermediate 15 was saponified by treatment with sodium hydroxide in a solvent mixture such as ethanol and THF at an elevated temperature such as 50° C. and for 15 h. This process resulted in the formation of intermediate 16 which may also be presented as a zwitterion.

Next, intermediate 15 is then reacted with commercially available pegylation reagents such as amine protected 17 using amide bond forming reaction conditions that are well known to those skilled in the art as shown in Scheme 3:

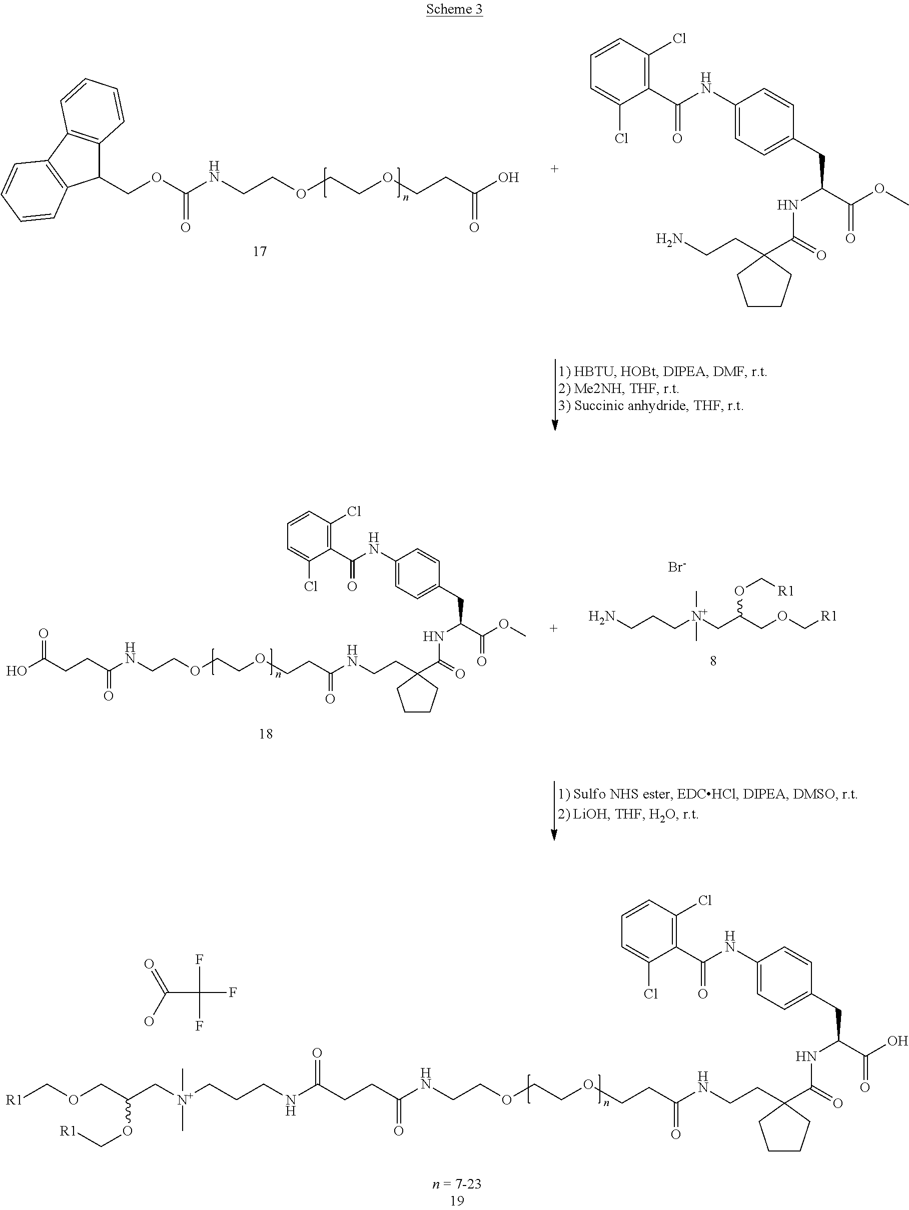

For compounds of general structure 17, different PEG lengths are available or easily made by those skilled in the art; specifically, n=8-24. The use of 9H-fluoren-9-ylmethoxycarbony protection (N-Fmoc) of the amino terminus of the pegylation reaction is preferred. The N-Fmoc protecting group is removed by treatment with a secondary amine such as dimethylamine in THF or piperidine in DMF (dimethylformamide). Having deprotected the amino terminus of the pegylated conjugate 18, reaction with commercially available succinic anhydride is performed as a means to expose a carboxylic acid group for subsequent coupling to the amino terminus to lipid intermediate 8, using amide bond forming reaction conditions that are well known to those skilled in the art. A preferred method is the use of hydroxy-2,5-dioxo-pyrrolidine-3-sulfonicacid (Sulfo-NHS) as the catalyst for a coupling mediated by 1-ethyl-3-(3-dimethyl]aminopropyl)carbodiimide hydrochloride (EDC-HCl) in the presence of a base such as di-isopropylethylamine (DIPEA) and in a solvent such as dimethylsulfoxide (DMSO). In this manner, after purification by reversed phased HPLC using elution systems containing trifluoroacetic acid salt (TFA) one is able to generate compounds such as 19 (TFA salt) which can easily be converted to compound 1 by treatment with base or basic ion exchange resin; such salt transformations are well known to those skilled in the art.

As shown in Scheme 4, it is possible to make compound 21 having a similar structure to compound 1 in a one-pot procedure by reacting intermediate 16 with commercially available PEG reagent 20 (Quanta Biodesign Limited) and lipid amine 8 in the presence of a base in aprotic solvent, such as dimethylsulfoxide at RT. After purification by HPLC, the TFA salt of a structure such as 21 (c=8 to 20) was obtained.

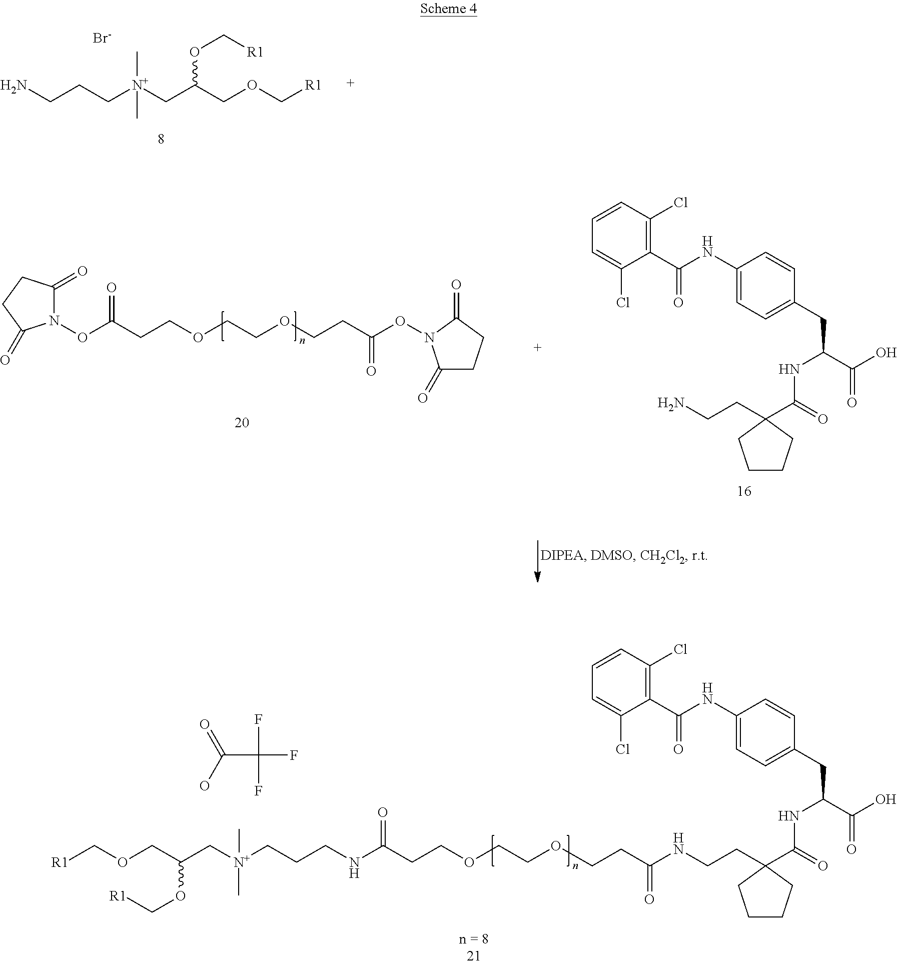

The synthesis of a compound of formula I:

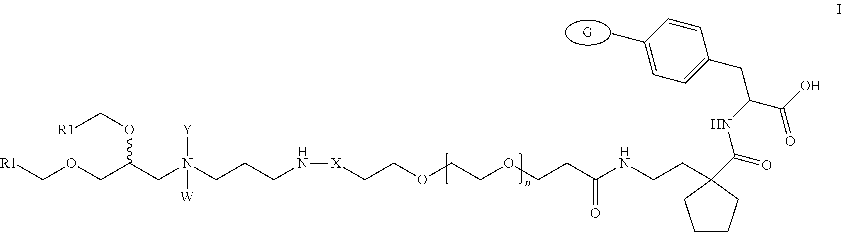

wherein:

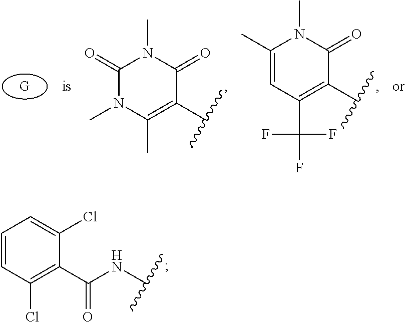

can be efficiently accomplished in accordance with Scheme 6:

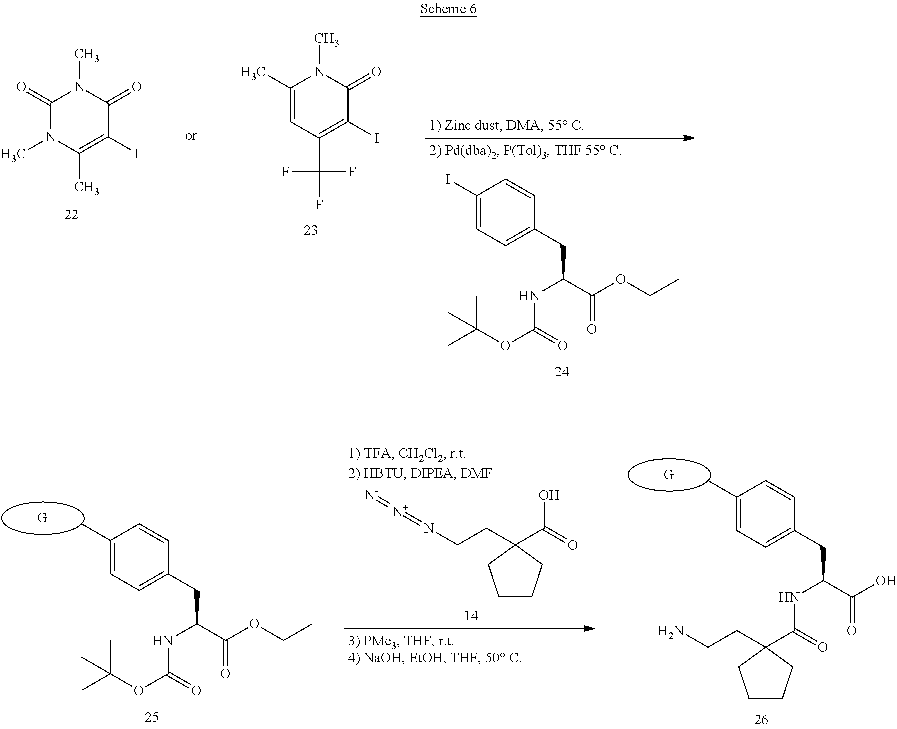 [SMILES: Cc1c(I)c(=O)n(C)c(=O)n1C ; Cc1cc(C(F)(F)F)c(I)c(=O)n1C ; CCOC(=O)[C@H](Cc1ccc(I)cc1)NC(=O)OC(C)(C)C ; CCOC(=O)[C@H](Cc1ccc(C)cc1)NC(=O)OC(C)(C)C ; [N-]=[N+]=NCCC1(C(=O)O)CCCC1 ; Cc1ccc(C[C@H](NC(=O)C2(CCN)CCCC2)C(=O)O)cc1]

Intermediate 26 is then made available for subsequent coupling in the same manner as is shown for intermediate 16 in Schemes 3 or 4 to produce the compounds of formula I. Detailed synthesis methods of the preparation of intermediate 26 in Scheme 6 are published in references U.S. Pat. Nos. 6,388,084 B1 and 6,380,387 B1. Briefly, aryl or heteroaryl zinc reagents are formed from known intermediates 22 or 23 in an anhydrous solvent such as dimethyl acetamide (DMA). At this point the zinc reagents are reacted with commercially available (S)—N-tert-butoxycarbonyl-(4-iodo)-phenylalanine ethyl ester in the presence of a palladium catalyst such as Pd(dba)₂ and in presence of palladium ligands tri-toluoylphosphine in an aprotic solvent such as THF at 50° C. In this manner, an intermediate of general structure 25 is formed. Intermediate 25 is then transformed to intermediate 26 in four steps which are routine to those skill in the art. First, the removal of the amino protecting group N-tert-butoxycarbonyl is effected in presence of strong acid such as TFA in DCM (CH₂Cl₂) solvent. Second, cyclic intermediate 14 is coupled to the amino group revealed in the previous step using standard amide bond forming conditions well known to those skill in the art. Third, the azido group is reduced to the corresponding amine using trimethyl phosphine in THF and finally the carboxylic ester is saponified using sodium hydroxide in a solvent mixture of THF and ethyl alcohol (EtOH) at 50° C. for 15 h. In this manner, as stated above, intermediate 26 is made available for subsequent coupling in the same manner as is shown for intermediate 16 in Schemes 3 or 4 to produce the compounds of formula I.

Compounds of formula I wherein:

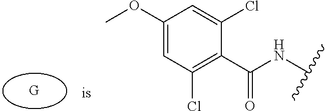 [SMILES: *NC(=O)c1c(Cl)cc(OC)cc1Cl]

can be also synthesized by the method as shown in Scheme 2.

### General Synthesis of Lipid Nanoparticles on the Invention

The lipid nanoparticles containing compounds of formula I can be prepared using an ethanol injection method as follows. The lipid portion of the nanoparticles are dissolved in absolute ethanol (200 proof) including:

(1) a compound selected from the group consisting of:
2-(2,2-di((9Z,12Z)-octadeca-9,12-dienyl)-1,3-dioxolan-4-yl)-N,N-dimethylethanamine;
N-(chloromethyl)-2-(2,2-di((9Z,12Z)-octadeca-9,12-dienyl)-1,3-dioxolan-4-yl)-N,N-dimethylethanamonium chloride;
2-(2,2-di((9Z,12Z)-octadeca-9,12-dienyl)-1,3-dioxolan-4-yl)-N,N,N-trimethyl-ethanamonium iodide; and
2-(2,2-di((9Z,12Z)-octadeca-9,12-dienyl)-1,3-dioxolan-4-yl)-N-ethyl-N,N-dimethyl-ethanamonium iodide;

(2) a compound of formula I (a VLA-4-targeting lipid)

(3) a phospholipid selected from the group consisting of:
1,2-dipalmitoyl-sn-glycero-3-phosphocholine,
1,2-distearoyl-sn-glycero-3-phosphocholine, and
1,2-dioleoyl-sn-glycero-3-phosphocholine; and

(4) cholesterol;

In a separate vial, hyaluronic acid and the polynucleotide were diluted to an appropriate concentration (i.e., 0.05-2 mg/mL for each hyaluronic acid and siRNA) using either nuclease-free water, citrate buffer, or dextrose (i.e., 5% or D5W). The lipid-ethanol solution was then added with rapid stirring to the aqueous siRNA containing solution resulting in a final ethanol concentration of 10-50%. After standing at RT for 3-24 h, under argon and shielded from the light, the formed lipid nanoparticle suspension was filtered using a 0.22 micron syringe filter (Millex-GV, Millipore) prior to use.

### Utility

The compounds of formula I are useful in formulating compositions to improve the delivery of therapeutic agents such as small molecules or nucleic acids (i.e. siRNA) to target cells expressing VLA-4.

Accordingly, the compositions of the present invention containing compounds of formula I may be used to encapsulate (or otherwise formulate) therapeutic agents such as small molecules or nucleic acids (i.e., siRNAs), for treating various diseases and conditions that are associated with the expression of VLA-4. Such disease and conditions include cancer and various metabolic related diseases.

In particular embodiments, the present invention comprises a method of treating or preventing cancer in a mammal (preferably a human) in need of such treatment, wherein the method comprises administering a therapeutically effective amount of a composition containing a compound of formula I. In particular embodiments such compositions are lipid nanoparticles. Such compositions can be administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations as the minimum amount necessary to inhibit the expression of the target protein and avoid unacceptable toxicity. For example, such amount may be below the amount that is toxic to normal cells, or the mammal as a whole. The compositions containing a compound of formula I of the invention may be administered by parenteral, intraperitoneal, and intrapulmonary administration. Parenteral infusions include intramuscular, intravenous, intraarterial, intraperitoneal, or subcutaneous administration.

### EXAMPLES

The invention will be more fully understood by reference to the following examples. They should not, however, be construed as limiting the scope of the invention.

Reagents were purchased from Aldrich, Sigma, and Pierce BioScience or other suppliers as indicated below and used without further purification. Reactions using microwave irradiation for heating were conducted using either a Personal Chemistry Emrys Optimizer System or a CEM Discovery System. The purification of multi-milligram to multi-gram scale was conducted by methods known to those skilled in the art such as elution of silica gel flash column. Preparative flash column purifications were also effected in some cases by use of disposable pre-packed multigram silica gel columns (RediSep) eluted with a CombiFlash system. Biotage™ and ISCO™ are also flash column instruments that may be used in this invention for purification of intermediates.

For the purpose of judging compound identity and purity, LC/MS (liquid chromatography/mass spectroscopy) spectra were recorded using the following system. For measurement of mass spectra, the system consists of a Micromass Platform II spectrometer: ES Ionization in positive mode (mass range: 150-1200 amu). The simultaneous chromatographic separation was achieved with the following HPLC system: ES Industries Chromegabond WR C-18 3u 120 Å (3.2×30 mm) column cartridge; Mobile Phase A: Water (0.02% TFA) and Phase B: Acetonitrile (0.02% TFA); gradient 10% B to 90% B in 3 min; equilibration time of 1 min; flow rate of 2 mL/minute. In some cases, ammonium acetate at 20 millimolar concentration was used as a modifier for effective ionization during preparative HPLC. In such cases, the ammonium salt was isolated.

For some separations, the use of super critical fluid chromatography may also be useful. Super critical fluid chromatography separations were performed using a Mettler-Toledo Minigram system with the following typical conditions: 100 bar, 30° C., 2.0 mL/min eluting a 12 mm AD column with 40% MeOH in super critical fluid CO₂. In the case of analytes with basic amino groups, 0.2% isopropyl amine was added to the methanol modifier.

Compounds were characterized either by ¹H-NMR using a Varian Inova 400 MHz NMR Spectrometer or a Varian Mercury 300 MHz NMR Spectrometer as well as by high resolution mass spectrometry using a Bruker Apex-II high-resolution 4.7T FT-Mass Spectrometer. Final compounds were also characterized by high resolution mass spectrometry using a LTQ CL Orbitrap sold by Thermo Electron.

Abbreviations used herein are as follows:
AIBN 2,2′-azobisisobutyronitrile
Bu butyl
DCE 1,2-dichloroethane
DCM dichloromethane
DIPEA diisopropylethylamine
DMF N,N-dimethylformamide
DMSO dimethylsulfoxide
EtOAc ethyl acetate
EtOH ethyl alcohol
FCC flash column chromatography
h hour(s)
HPLC high pressure liquid chromatography
HRMS high resolution mass spectra
LRMS low resolution mass spectra
LC liquid chromatography
L-Pro L-proline
MCPBA meta-chloroperoxybenzoic acid
MeOH methyl alcohol
MW molecular weight
NIS N-iodosuccinimide
NB S N-bromosuccinimide
NMP 1-methyl-2-pyrrolidinone
PdCl₂(dppf) [1,1′-Bis(diphenylphosphino)ferrocene]dichloropalladium(II)
PG protecting group
PyBroP bromo-tris-pyrrolidino-phosphonium hexafluorophosphate
RT room temperature
TBAF tetrabutylammonium fluoride
TBDMS tert-butyl-dimethylsilyl
TBTU 2-(1H-benzotriazole-1-yl)-1,1,3,3-tetramethyluronium tetrafluoroborate
TMS trimethylsilyl
TMSSMe (methylthio)trimethylsilane
TEA triethylamine
TEMPO 2,2,6,6-tetramethylpiperidine-1-oxyl
TFA trifluoroacetic acid
THF tetrahydrofuran

### Preparation of Intermediates

### Preparation of 4-[(2,6-dichlorophenylcarbonyl)amino]-N—[[1-(2-aminoethyl)cyclopentyl]carbonyl]-L-phenylalanine

### a) Preparation of 1-(2-bromoethyl)cyclopentane carboxylic acid methyl ester

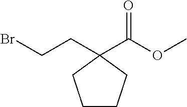

To a solution of diisopropylamine (396 mmol, 56 mL) in THF (85 mL) was added dropwise a solution of n-butyllithium (393 mmol, 240 mL, 1.6M) in hexanes at −10° C. while maintaining the temperature below 0° C. After addition, the solution was stirred for 30 min at 0° C. To this, a solution of cyclopentane carboxylic acid methyl ester (263 mmol, 37.4 g) in THF (50 mL) was added dropwise at −70° C. maintaining the internal temperature between −60 and −70° C. After addition, the reaction mixture was stirred for 1 h at −50 to −60° C. Then, a solution of 1,2-dibromoethane (545 mmol, 47 mL) in THF (50 mL) was added dropwise and the light brown suspension was stirred for 1 h at −70 to −60° C. Then, it was allowed to warm to RT and stirred overnight. The reaction mixture was poured into a saturated aqueous solution of ammonium chloride (200 mL) and the organic compound was extracted into ether (2×100 mL). The combined extracts were washed with a saturated solution of sodium chloride (150 mL) and dried over anhydrous magnesium sulfate. After filtration of the drying agent, the solution was concentrated under vacuum and the resulting residue was distilled at 95-105° C./2.5 mm Hg to obtain 49.6 g (80% yield) of 1-(2-bromoethyl)cyclopentane carboxylic acid methyl ester as a colorless oil.

### b) Preparation of 1-[2-(azido)ethyl]cyclopentane carboxylic acid methyl ester

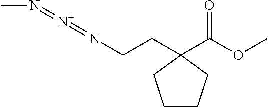

A solution of 1-(2-bromoethyl)cyclopentane carboxylic acid methyl ester (211 mmol, 49.6 g) and sodium azide (831 mmol, 54 g) in DMF (200 mL) was stirred at 50° C. for 5 h under nitrogen atmosphere. Then, the solids were filtered and the filtrate was concentrated to near dryness. The residue was diluted with ethyl acetate (500 mL) and the undissolved solids were collected by filtration and the filtrate was concentrated to give the crude ethyl 1-(2-azidoethyl)cyclopentane carboxylate which was purified by chromatography over 250 g of silica gel, eluting with 5% ethyl acetate in hexane to give 36.2 g (87% yield) of 1-[2-(azido)ethyl]cyclopentane carboxylic acid methyl ester as a light brown oil. EI(+)—HRMS m/e calcd. For C₉H₁₅N₃O₂ (M−H)⁺ 196.1086, obsd. 196.1342.

### c) Preparation of 1-[2-(azido)ethyl]cyclopentane carboxylic acid

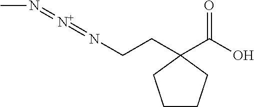

The 1-[2-(azido)ethyl]cyclopentane carboxylic acid methyl ester (184 mmol, 36.2 g) was dissolved in THF (500 mL) and methanol (250 mL) and a solution of LiOH monohydrate (368 mmol, 15.44 g) in water (300 mL) was added. The resulting solution was stirred at 40° C. overnight and concentrated. The residue was dissolved in 1 L of water containing 40 mL of 1N NaOH and was washed with hexane (500 mL). The aqueous layer was acidified with 1N HCl and the organic compound was extracted with ether (2×500 mL). The combined extracts were washed with saturated sodium chloride solution and the organic layer was dried over anhydrous Na₂SO₄. Filtration of the drying agent and concentration gave 32.5 g (96% yield) of 1-[2-(azido)ethyl]cyclopentane carboxylic acid as an amber liquid. ES(+)-HRMS m/e calcd. for C₈H₁₃N₃O₂ (M+Na)⁺ 206.0900, obsd. 206.0900.

### d) Preparation of (S)-2-tert-butoxycarbonylamino-3-(4-nitro-phenyl)-propionic acid methyl ester

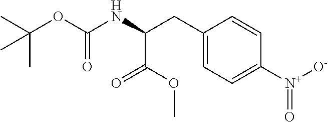 [SMILES: COC(=O)[C@H](Cc1ccc([N+](=O)[O-])cc1)NC(=O)OC(C)(C)C]

To a suspension of (S)-2-tert-butoxycarbonylamino-3-(4-nitro-phenyl)-propionic acid (226.2 mmol, 70.2 g) and sodium carbonate (1.13 mol, 95 g) in DMF (500 mL) was added methyl iodide (1.13 mol, 70.4 mL) at RT. The suspension was stirred for 15 h at RT at which time TLC analysis of the mixture indicated the absence of starting material and the excess methyl iodide and some DMF were removed under high vacuum. Then, it was poured into water (2 L) and stirred at RT as a precipitate formed slowly over 72 h. The precipitated solids were collected by filtration and washed with water (2 L). After air and vacuum drying, 72 g (98% yield) of (S)-2-tert-butoxycarbonylamino-3-(4-nitro-phenyl)-propionic acid methyl ester was isolated as a light yellow solid (mp 95-96° C.). ES(+)-HRMS m/e calcd. for C₁₅H₂₀N₂O₆ (M+H)⁺ 325.1400, obsd. 325.1404.

### e) Preparation of (S)-3-(4-amino-phenyl)-2-tert-butoxycarbonylamino-propionic acid methyl ester

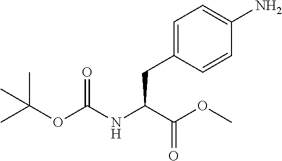 [SMILES: COC(=O)[C@H](Cc1ccc(N)cc1)NC(=O)OC(C)(C)C]

To a mixture of (S)-2-tert-butoxycarbonylamino-3-(4-nitro-phenyl)-propionic acid methyl ester (222 mmol, 72 g), zinc dust (325 mesh, 2.2 mol, 145.2 g, 10 equiv.) and ammonium chloride (3.3 mol, 178.1 g, 15 equiv.) was added methanol (1 L) and water (500 mL) at RT. After addition of water, an exothermic reaction ensued and the internal temperature rose to 45-50° C. The suspension was stirred for 30 min to 1 h at RT, at which time TLC analysis of the mixture indicated the absence of starting material, and the reaction mixture was filtered through a pad of celite and the filtered cake was washed with methanol (1 L) and water (500 mL). Concentration to remove most of the methanol and some water afforded white solids which were collected by filtration and washed with water. After air drying, 65.5 g (100% yield) of (S)-3-(4-amino-phenyl)-2-tert-butoxycarbonylamino-propionic acid methyl ester was isolated as a white solid (mp 86-89° C.). ES(+)-HRMS m/e calcd. for C₁₅H₂₂N₂O₄ (M+H)⁺ 294.1621, obsd. 294.1614.

### f) Preparation of (S)-2-tert-butoxycarbonylamino-3-[4-(2,6-dichloro-benzoylamino)-phenyl]-propionic acid methyl ester

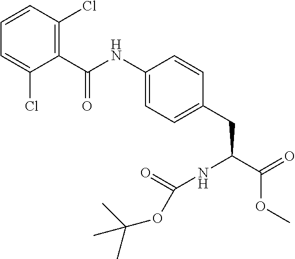 [SMILES: COC(=O)[C@H](Cc1ccc(NC(=O)c2c(Cl)cccc2Cl)cc1)NC(=O)OC(C)(C)C]

To a solution of (S)-3-(4-amino-phenyl)-tert-butoxycarbonylamino-propionic acid methyl ester (127.6 mmol, 37.57 g) and 2,6-dichlorobenzoyl chloride (140.6 mmol, 29.45 g) in dichloromethane (350 mL) was added DIPEA (192 mmol, 24.8 g) at RT. The brown solution was stirred for 15 h at RT to afford a white suspension. At this time TLC analysis of the mixture indicated the absence of starting material. Then, the solids were collected by filtration and the solids were washed with dichloromethane (150 mL) and air dried to obtain 52.75 g (88.4% yield) of (S)-2-tert-butoxycarbonylamino-3-[4-(2,6-dichloro-benzoylamino)-phenyl]-propionic acid methyl ester as a white solid: mp 192-194° C. ES(+)-HRMS m/e calcd. for C₂₂H₂₄Cl₂N₂O₅ (M+H)⁺ 466.1062, obsd. 466.1069.

### g) Preparation of (S)-2-amino-3-[4-(2,6-dichloro-benzoylamino)-phenyl]-propionic acid methyl ester hydrochloride salt

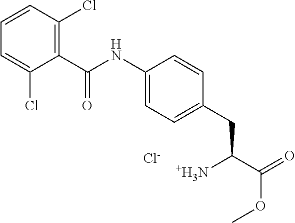 [SMILES: COC(=O)[C@@H]([NH3+])Cc1ccc(NC(=O)c2c(Cl)cccc2Cl)cc1 ; [Cl-]]

The solid S)-2-tert-butoxycarbonylamino-3-[4-(2,6-dichloro-benzoylamino)-phenyl]-propionic acid methyl ester (92.97 mmol, 43.45 g) in dioxane (90 mL) was treated with 166 mL of 4.0N HCl in dioxane at RT. After 5 minutes, the solids went into solution and the mixture was stirred for 2 h. Then, some of the dioxane was removed under vacuum to afford an yellow syrup and 250 mL of ethyl ether was added. A gum was formed which was dissolved in THF (100 mL) and methanol (100 mL). The solvent was removed under vacuum to obtain 43.7 g (100% yield) of (S)-2-amino-3-[4-(2,6-dichloro-benzoylamino)-phenyl]-propionic acid methyl ester hydrochloride salt as a white solid: mp 180-195° C. This was stored in the refrigerator under argon atmosphere. ES(+)-HRMS m/e calcd. for C₁₇H₁₆Cl₂N₂O₃ (M+H)⁺ 367.0616, obsd. 367.0611.

### h) Preparation of (S)-2-[[1-(2-azido-ethyl)-cyclopentanecarbonyl]-amino]-3-[4-(2,6-dichloro-benzoylamino)-phenyl]-propionic acid methyl ester

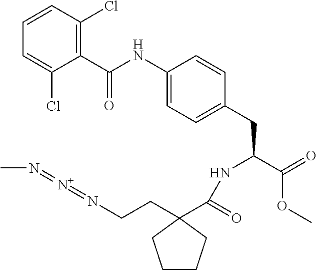

To a solution of (S)-2-amino-3-[4-(2,6-dichloro-benzoylamino)-phenyl]-propionic acid methyl ester hydrochloride salt (24.79 mmol, 10 g) and 1-[2-(azido)ethyl]cyclopentane carboxylic acid (27.29 mmol, 5 g) in DMF (100 mL) were added HBTU (27.33 mmol, 10.37 g) and DIPEA (68.3 mmol, 8.82 g) at RT. The clear solution was stirred for 48 h at RT and water was added (200 mL) to the reaction mixture to precipitate the product. The solid was collected by filtration and washed with water (100 mL) and hexane (100 mL). After drying at air, 11.2 g of the product was obtained as a light brick solid which was treated with acetonitrile (100 mL) at hot condition. All impurities went into acetonitrile and the solid was collected by filtration to afford 8.24 g of coupling product. The acetonitrile solution was removed under vacuum and the residue was dissolved in ethyl acetate and the product was precipitated by the addition of hexane. Again, the solid was collected by filtration and the solids were dried at air to obtain another 2.03 g (78% total yield) of(S)-2-[[1-(2-azido-ethyl)-cyclopentanecarbonyl]-amino]-3-[4-(2,6-dichloro-benzoylamino)-phenyl]-propionic acid methyl ester as a white solid. ES(+)-HRMS m/e calcd. for C₂₅H₂₇Cl₂N₅O₄ (M+Na)⁺ 554.1332, obsd. 554.1334.

### i) Preparation of (S)-2-[[1-(2-azido-ethyl)-cyclopentanecarbonyl]-amino]-3-[4-(2,6-dichloro-benzoylamino)-phenyl]-propionic acid

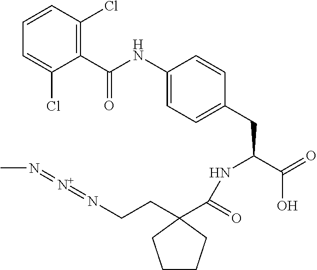 [SMILES: CN=[N+]=NCCC1(C(=O)N[C@@H](Cc2ccc(NC(=O)c3c(Cl)cccc3Cl)cc2)C(=O)O)CCCC1]

To a suspension of (S)-2-[[1-(2-azido-ethyl)-cyclopentanecarbonyl]-amino]-3-[4-(2,6-dichloro-benzoylamino)-phenyl]-propionic acid methyl ester (27.7 mmol, 14.77 g) in THF (200 mL) and ethanol (200 mL) was added aqueous 1.0 N sodium hydroxide (150 mL) at RT. The mixture was stirred for 15 h at RT at which time TLC analysis of the mixture indicated the absence of starting material. Then, it was diluted with water (20 mL) and the THF and ethanol was removed by rotary evaporation and diluted with 100 mL of water and extracted with ether (200 mL) to remove any neutral impurities. The aqueous layer was neutralized with 1 N HCl and the resulting white solids were collected by filtration and washed with water and hexanes. After air-drying, 13.37 g (93% yield) of(S)-2-[[1-(2-azido-ethyl)-cyclopentane-carbonyl]-amino]-3-[4-(2,6-dichloro-benzoylamino)-phenyl]-propionic acid was obtained as a white solid. ES(+)-HRMS m/e calcd. for C₂₄H₂₅Cl₂N₅O₄ (M+Na)⁺ 540.1176, obsd. 540.1177.

### j) Preparation of (S)-2-[[1-(2-amino-ethyl)-cyclopentanecarbonyl]-amino]-3-[4-(2,6-dichloro-benzoylamino)-phenyl]-propionic acid trifluoroacetate salt

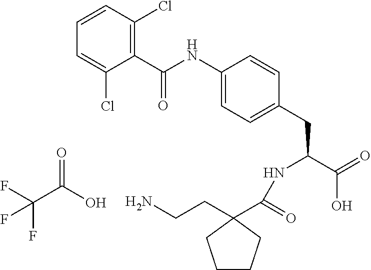 [SMILES: O=C(O)C(F)(F)F ; NCCC1(C(=O)N[C@@H](Cc2ccc(NC(=O)c3c(Cl)cccc3Cl)cc2)C(=O)O)CCCC1]

To a solution of (S)-2-[[1-(2-azido-ethyl)-cyclopentanecarbonyl]-amino]-3-[4-(2,6-dichloro-benzoylamino)-phenyl]-propionic acid (12.11 mmol, 6.28 g) in THF (91 mL) was added a solution of trimethylphosphine in THF (48.46 mmol, 48.5 mL, 1.0 NJ) at 0° C. It was a clear solution in the beginning and after 30 min some precipitate was formed and this mixture was stirred for overnight at which time TLC analysis of the mixture indicated the absence of starting material. Then, 10 equiv. of water (120 mmol, 2.16 mL) was added and the mixture was stirred for 2 h at RT. The solvent was removed under vacuum and the residue was azeotrophed two times with toluene to give a pasty material which was purified using HPLC method to obtain 4.57 g (77% yield) of (S)-2-[[1-(2-amino-ethyl)-cyclopentanecarbonyl]-amino]-3-[4-(2,6-dichloro-benzoylamino)-phenyl]-propionic acid trifluoroacetate salt as an amorphous white solid. ES(+)-HRMS m/e calcd. for C₂₄H₂₇Cl₂N₃O₄ (M+H)⁺ 492.1452, obsd. 492.1451.

### General Method 1: Preparation of (rac)-N-(3-aminopropyl)-2,3-bis(dodecyloxy)-N,N-dimethyl-1-propanaminium bromide (GAP-DLRIE)

### a) Preparation of Methanesulfonic Acid Dodecyl Ester

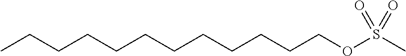 [SMILES: CCCCCCCCCCCCOS(C)(=O)=O]

To a solution of dodecan-1-ol (200 mmol, 37.27 g) and triethylamine (460 mmol, 64.1 mL) in dichloromethane (300 mL) was added dropwise methanesulfonyl chloride (240 mmol, 27.5 g) at 0° C. for 30 minutes under nitrogen atmosphere. The resulting light brown suspension was stirred for 1.5 h at this temperature and then the cooling bath was removed to allow the reaction mixture to warm to RT during 30 minutes. Then, a saturated solution of ammonium chloride (300 mL) was added and the two layers were separated. The aqueous layer was extracted with dichloromethane (200 mL) and the combined organic layer was washed with water (300 mL) and brine solution (300 mL). The organic layer was dried over anhydrous magnesium sulfate, filtration and concentration gave the crude light brown oil which was dissolved in ethyl acetate (50 mL) at hot condition and then diluted with hexanes (100 mL). The clear solution was stored in the refrigerator for 15 h. The resulting off-white solids were collected by filtration and washed with hexanes. After air drying, 45.53 g (86% yield) of methanesulfonic acid dodecyl ester was isolated as an off-white solid. ES(+)-LRMS m/e calcd. for C₁₃H₂₈O₃S (M+H)⁺ 264.1759, obsd. 265.0.

### b) Preparation of (rac)-2,3-bis(dodecyloxy-propyl)-N,N-dimethyl-amine

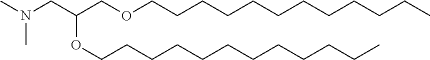 [SMILES: CCCCCCCCCCCCOCC(CN(C)C)OCCCCCCCCCCCC]

Sodium hydride (38.6 mmol, 1.54 g, 60% in oil) was charged in a 250 mL two-neck RB flask under nitrogen atmosphere and anhydrous THF (20 mL) was added to remove oil. The resulting suspension was stirred for 10 minutes and then the stirring was stopped to settle the sodium hydride solids. The supernatant liquid was removed with syringe and a fresh THF (100 mL) was added. To this slurry, a solution of 3-(dimethylamino)-propane-1,2-diol (14.9 mmol, 1.77 g) in THF (15 mL) was added dropwise for 10 minutes at RT. The resulting suspension was heated to reflux for 24 h. Then, a solution of methanesulfonic acid dodecyl ester (38.6 mmol, 10.2 g) in THF (22 mL) was added and again the mixture was heated to reflux for 5 days. Then, it was cooled to RT and the mixture was filtered through a 3-cm plug of celite while washing with ethyl acetate (1.0 L). The filtrate was removed under vacuum and the residue was partitioned between diethyl ether (250 mL) and 0.2M aqueous sodium hydroxide solution (250 mL). The two layers were separated and the organic layer was washed with water (200 mL), brine solution (200 mL), and dried over anhydrous magnesium sulfate. Filtration and concentration gave the light brown oil (7.93 g) which was purified using an ISCO (150 g) flash column chromatography to obtain 4.51 g (67% yield) of (rac)-2,3-bis(dodecyloxy-propyl)-N,N-dimethyl-amine as a light yellow oil. ES(+)-HRMS m/e calcd. for C₂₉H₆₁NO₂ (M+H)⁺ 456.4775, obsd. 456.4785.

### c) Preparation of (rac)-N-[2,3-bis(dodecyloxy)propyl]-1,3-dihyro-N,N-dimethyl-1,3-dioxo-2H-isoindole-2-propanaminium bromide

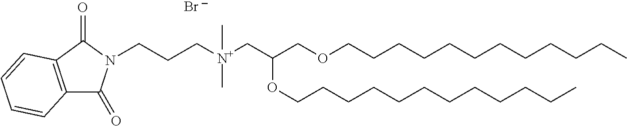 [SMILES: [Br-] ; CCCCCCCCCCCCOCC(C[N+](C)(C)CCCN1C(=O)c2ccccc2C1=O)OCCCCCCCCCCCC]

To a mixture of 2-(3-bromopropyl)-isoindoline-1,3-dione (8.37 mmol, 2.24 g) and (rac)-2,3-bis(dodecyloxy-propyl)-N,N-dimethyl-amine (4.48 mmol, 2.04 g) was added DMF (8.5 mL) at RT under nitrogen atmosphere. The resulting solution was heated to 107° C. and stirred for 4 days. Then, it was cooled to RT and the DMF was removed under vacuum and then the residue was purified using HPLC method to obtain 2.4 g (83% yield) of (rac)-N-[2,3-bis(dodecyloxy)propyl]-1,3-dihyro-N,N-dimethyl-1,3-dioxo-2H-isoindole-2-propanaminium bromide as a white paste. ES(+)-LRMS m/e calcd. for C₄₀H₇₁N₂O₄ (M+H)⁺ 643.54, obsd. 643.4.

### d) Preparation of (rac)-N-(3-aminopropyl)-2,3-bis(dodecyloxy)-N,N-dimethyl-1-propanaminium bromide

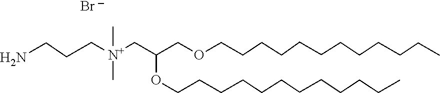 [SMILES: [Br-] ; CCCCCCCCCCCCOCC(C[N+](C)(C)CCCN)OCCCCCCCCCCCC]

To a clear solution of (rac)-N-[2,3-bis(dodecyloxy)propyl]-1,3-dihyro-N,N-dimethyl-1,3-dioxo-2H-isoindole-2-propanaminium bromide (3.73 mmol, 2.4 g) in anhydrous ethanol (40 mL) was added an excess of anhydrous hydrazine (130 mmol, 4.1 mL) at RT under nitrogen atmosphere. Then, the nitrogen line was disconnected and the flask was sealed with a cap and the reaction mixture was stirred for 3 days by which time lot of white precipitate was formed. Then, the solids were collected by filtration using filter paper and the solids were washed with dichloromethane (˜200 mL). The filtrate was removed under vacuum and the residue was dissolved in dichloromethane (100 mL) and it was washed with 0.1N aqueous NaOH solution. The aqueous basic layer was extracted with dichloromethane (100 mL) and the combined organic layer was washed with brine solution (lot of precipitates were formed after addition of brine solution). The organic layer was dried over anhydrous magnesium sulfate and filtration, concentration gave 1.47 g (77% yield) of (rac)-N-(3-aminopropyl)-2,3-bis(dodecyloxy)-N,N-dimethyl-1-propanaminium bromide as a white solid. ES(+)-LRMS m/e calcd. for C₃₂H₇₀N₂O₂ (M+H)⁺ 513.5, obsd. 513.0.

### Example 1

### General method 2: Preparation of [2,3-bis(dodecyloxy)propyl][3-[[3-[2-[2-[2-[2-[2-[2-[2-[2-[[3-[[2-[1-[[[(S)-1-carboxy-2,4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate

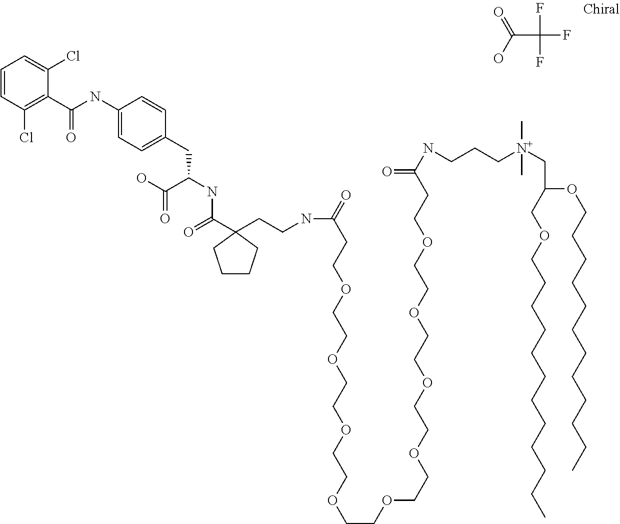 [SMILES: CCCCCCCCCCCCOCC(C[N+](C)(C)CCCNC(=O)CCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCC ; O=C(O)C(F)(F)F]

To a clear solution of bis-(2,5-dioxopyrrolidin-1-yl)-4,7,10,13,16,19,22,25,28-nonaoxahentria-contane-1,31-dioate (0.141 mmol, 100 mg) in DMSO (2 mL) was added dropwise a solution of (S)-2-[[1-(2-amino-ethyl)-cyclopentanecarbonyl]-amino]-3-[4-(2,6-dichloro-benzoylamino)-phenyl]-propionic acid trifluoroacetate salt (0.141 mmol, 69.5 mg) and DIPEA (0.423 mmol, 75 uL) in DMSO (3 mL) for 30 minutes at RT under nitrogen atmosphere. The resulting reaction mixture was stirred for 1 h at which time LCMS analysis indicated the presence of ˜62% of the mono coupling intermediate (mass: 1083). Then, the solid N-(3-aminopropyl)-2,3-bis(dodecyloxy)-N,N-dimethylpropan-1-aminium bromide (0.169 mmol, 87 mg) was added followed by DIPEA (0.423 mmol, 55 mg) at RT. The resulting suspension was stirred for 2 h by which time the lipid was not soluble. Then, 2 mL of chloroform was added and the resulting clear solution was stirred for 15 h at RT under nitrogen atmosphere. Then, the chloroform and excess DIPEA was removed under vacuum and the desired product was isolated by purification using HPLC method to obtain 75 mg (36% yield) of [2,3-bis(dodecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate as a colorless paste. ES(+)-HRMS m/e calcd. for C₇₈H₁₃₄Cl₂N₅O₁₇ (M+)⁺ 1482.9147, obsd. 1482.9142.

### Preparation of 4-[[2-[2-[2-[2-[2-[2-[2-[2-[2-[2-[2-[2-[[3-[[2-[1-[[[(S)-1-(methoxy-carbonyl)-2-[4-(2,6-dichlorobenzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-4-oxobutanoic acid

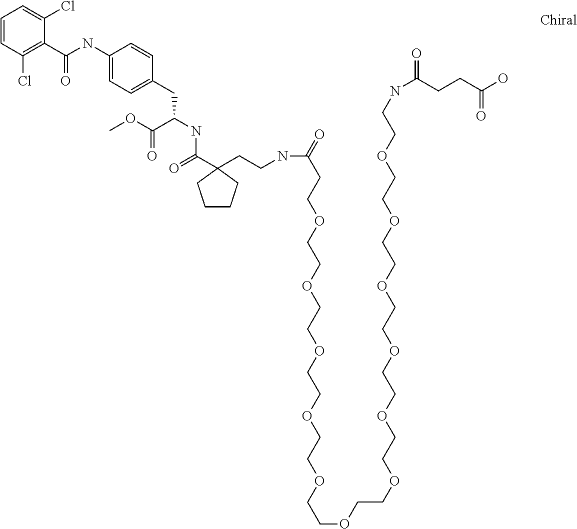 [SMILES: COC(=O)[C@H](Cc1ccc(NC(=O)c2c(Cl)cccc2Cl)cc1)NC(=O)C1(CCNC(=O)CCOCCOCCOCCOCCOCCOCCOCCOCCOCCOCCOCCOCCNC(=O)CCC(=O)O)CCCC1]

### a) Preparation of (S)-2-[[[1-(2-aminoethyl)cyclopentyl]carbonyl]amino]-3-[4-(2,6-dichloro-benzoylamino)phenyl]propionic acid methyl ester

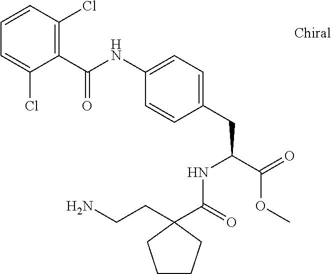 [SMILES: COC(=O)[C@H](Cc1ccc(NC(=O)c2c(Cl)cccc2Cl)cc1)NC(=O)C1(CCN)CCCC1]

To a solution of (S)-2-[1-(2-azidoethyl)cyclopentanecarbonyl-(amino)]-3-[4-(2,6-dichloro-benzoylamino)-phenyl]-propionic acid methyl ester (17.7 mmol, 9.42 g) in THF (110 mL) was added a solution of trimethylphosphine in THF (70.8 mmol, 70.8 mL, 1.0 Ai) at RT under nitrogen atmosphere. The resulting light brown solution was stirred for overnight at which time TLC analysis of the mixture indicated the absence of starting material. Then, 3 equiv. of water (53.1 mmol, 0.96 mL) was added and the mixture was stirred for 2 h at RT. The solvent was removed under vacuum and the residue was dissolve in THF (10 mL) and toluene (15 mL) and again the solvent was removed under vacuum. The resulting brown solid was dried under high vacuum to obtain an amorphous light brown solid which was triturated with hot hexanes and ethyl acetate. After cooling to room temperature, the resulting light brown solids were collected by filtration and washed with ethyl acetate. After drying under high vacuum, 8.9 g (99% yield) of (S)-2-[[[1-(2-amino ethyl)cyclopentyl]carbonyl]amino]-3-[4-(2,6-dichlorobenzoylamino)phenyl]propionic acid methyl ester was obtained as a light brown solid. ES(+)-LRMS m/e calcd. for C₂₅H₂₉Cl₂N₃O₄ (M+)⁺ 506.43, obsd. 506.2.

### b) Preparation of N-[2-[2-[2-[2-[2-[2-[2-[2-[2-[2-[2-[2-[[3-[[2-[1-[[[(S)-2-[4-(2,6-dichloro-benzoylamino)phenyl]-1-(methoxycarbonyl)ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]carbamic acid 9H-fluoren-9-ylmethyl ester

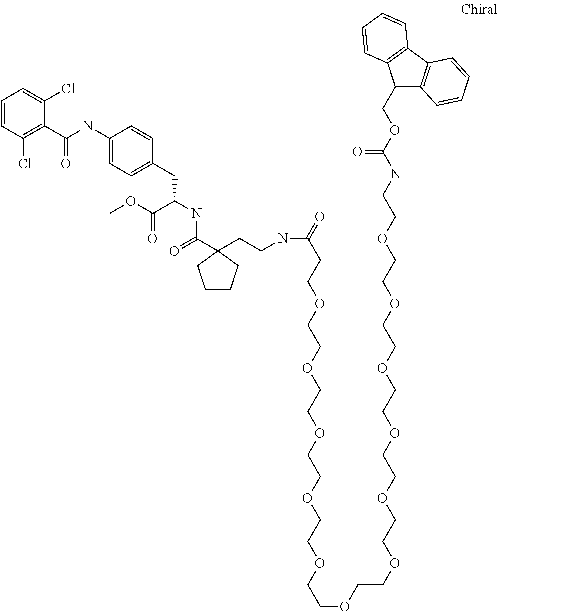 [SMILES: COC(=O)[C@H](Cc1ccc(NC(=O)c2c(Cl)cccc2Cl)cc1)NC(=O)C1(CCNC(=O)CCOCCOCCOCCOCCOCCOCCOCCOCCOCCOCCOCCOCCNC(=O)OCC2c3ccccc3-c3ccccc32)CCCC1]

To a suspension of (S)-2-[1-(2-aminoethyl)cyclopentanecarbonyl-(amino)]-3-[4-(2,6-dichloro-benzoylamino)-phenyl]-propionic acid methyl ester (1.43 mmol, 723 mg), 1-(9H-fluoren-9-yl)-3-oxo-2,7,10,13,16,19,22,25,28,31,34,37,40-tridecaoxa-4-azatritetracontan-43-oic acid (1.19 mmol, 1.0 g), HBTU (1.31 mmol, 497 mg), and 1-hydroxybenzotriazole (1.31 mmol, 177 mg) in DMF (20 mL) was added excess DIPEA (3.93 mmol, 508 mg) at RT under nitrogen atmosphere. After 20 min, it gave a clear solution which was stirred for 2 days at which time LCMS analysis of the mixture indicated the presence of the desired product. Then, water was added and the organic compound was extracted into ethyl acetate (3×50 mL). The combined extracts were washed with water (100 mL) and brine solution (100 mL). The organic layer was dried over anhydrous magnesium sulfate, filtration, and concentration gave the crude residue which was purified using HPLC method to obtain 360 mg (23% yield) of N-[2-[2-[2-[2-[2-[2-[2-[2-[2-[2-[2-[2-[[3-[[2-[1-[[[(S)-2-[4-(2,6-dichloro-benzoylamino)phenyl]-1-(methoxycarbonyl)ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]carbamic acid 9H-fluoren-9-ylmethyl ester as a light brown oil. ES(+)-LRMS m/e calcd. for C₆₇H₉₂Cl₂N₄O₁₉ (M+H)⁺ 1328.5733, obsd. 1329.3.

### c) Preparation of (S)-Alpha-[[[1-[2-[[3-[2-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethoxy]ethoxy]-1-oxopropyl]amino]ethyl]cyclopentyl]carbonyl]amino]-4-(2,6-dichlorobenzoylamino) benzenepropanoic acid methyl ester

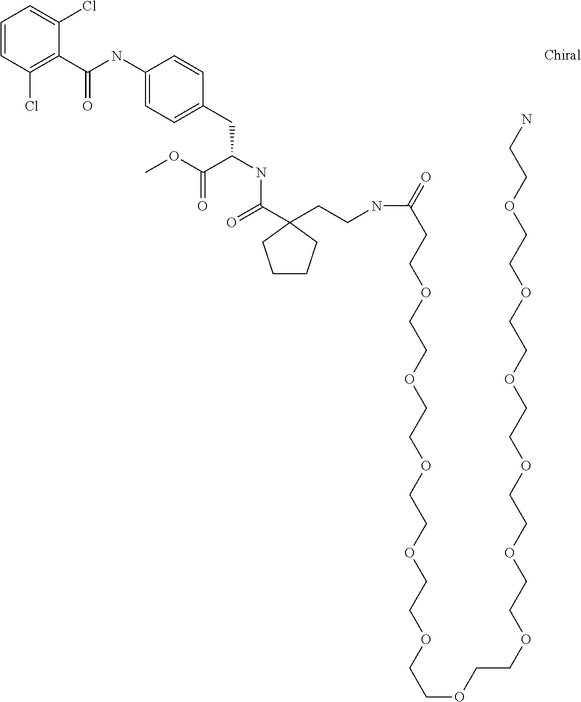 [SMILES: COC(=O)[C@H](Cc1ccc(NC(=O)c2c(Cl)cccc2Cl)cc1)NC(=O)C1(CCNC(=O)CCOCCOCCOCCOCCOCCOCCOCCOCCOCCOCCOCCOCCN)CCCC1]

To a solution of N-[2-[2-[2-[2-[2-[2-[2-[2-[2-[2-[2-[2-[[3-[[2-[1-[[[(S)-2-[4-(2,6-dichloro-benzoylamino)phenyl]-1-(methoxycarbonyl)ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]carbamic acid 9H-fluoren-9-ylmethyl ester (0.1 mmol, 132 mg) in DMF (5 mL) was added excess piperidine (2.0 mmol, 197 μL) at RT under nitrogen atmosphere. The resulting solution was stirred for 2 h at which time LCMS analysis of the mixture indicated the presence of the desired product. Then, the solvent was removed under high vacuum and the crude residue was purified using HPLC method to obtain 75 mg (68% yield) of (S)-Alpha-[[[1-[2-[[3-[2-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethoxy]ethoxy]-1-oxopropyl]amino]ethyl]cyclopentyl]carbonyl]amino]-4-(2,6-dichloro-benzoylamino)benzenepropanoic acid methyl ester as a light brown oil. ES(+)-LRMS m/e calcd. for C₅₂H₈₂Cl₂N₄O₁₇ (M+H)⁺ 1106.1559, obsd. 1107.0.

### d) Preparation of 4-[[2-[2-[2-[2-[2-[2-[2-[2-[2-[2-[2-[2-[[3-[[2-[1-[[[(S)-1-(methoxycarbonyl)-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-4-oxobutanoic acid

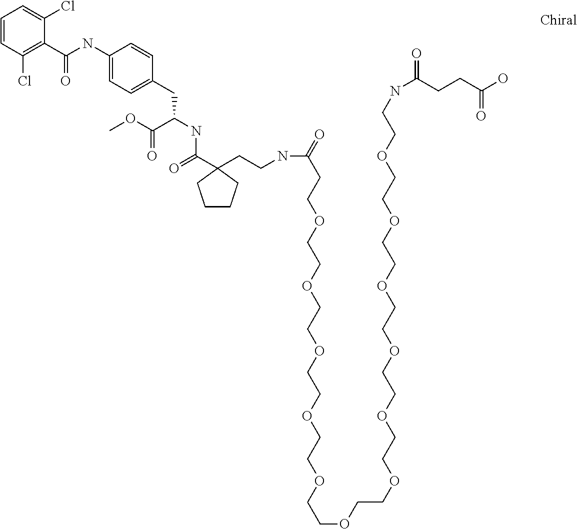 [SMILES: COC(=O)[C@H](Cc1ccc(NC(=O)c2c(Cl)cccc2Cl)cc1)NC(=O)C1(CCNC(=O)CCOCCOCCOCCOCCOCCOCCOCCOCCOCCOCCOCCOCCNC(=O)CCC(=O)O)CCCC1]

To a solution of (S)-Alpha-[[[1-[2-[[3-[2-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethoxy]ethoxy]-3-oxo-propyl]amino]ethyl]cyclopentyl]carbonyl]amino]-4-(2,6-dichlorobenzoylamino)benzene-propanoic acid methyl ester (0.062 mmol, 68 mg) and dihydrofuran-2,5-dione (0.062 mmol, 6.2 mg) in THF (3 mL) was added excess DIPEA (0.31 mmol, 40 mg) at RT under nitrogen atmosphere. The resulting colorless solution was stirred for 5 h at which time LCMS analysis of the mixture indicated the absence of starting material. Then, the solvent was removed under high vacuum and the crude residue was purified using an ISCO (12 g) column chromatography eluting with 0-50% methanol in dichloromethane to obtain 70 mg (94% yield) of 4-[[2-[2-[2-[2-[2-[2-[2-[2-[2-[2-[2-[2-[[3-[[2-[1-[[[(S)-1-(methoxycarbonyl)-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-4-oxobutanoic acid as a light brown oil. ES(+)-LRMS m/e calcd. for C₅₆H₈₆Cl₂N₄O₂₀ (M+H)⁺ 1206.2306, obsd. 1207.2.

### Example 2

### General Method 3: Preparation of [2,3-bis(dodecyloxy)propyl][3-[[4-[[2-[2-[2-[2-[2-[2-[2-[2-[2-[2-[2-[2-[[3-[[2-[1-[[[(S)-1-carboxy-2-[4-(2,6-dichlorobenzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1-4-dioxobutyl]amino]propyl 1 dimethyl-aminium trifluoroacetate

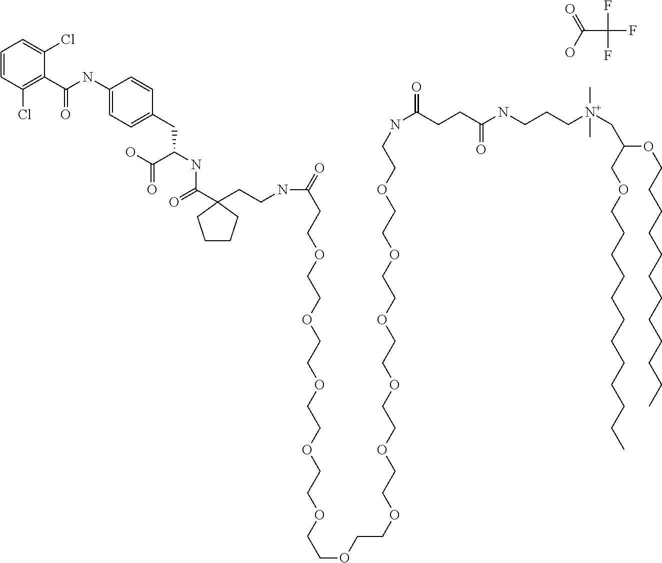 [SMILES: CCCCCCCCCCCCOCC(C[N+](C)(C)CCCNC(=O)CCC(=O)NCCOCCOCCOCCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCC ; O=C(O)C(F)(F)F]

### a) Preparation of [2,3-bis(dodecyloxy)propyl][3-[[4-[[2-[2-[2-[2-[2-[2-[2-[2-[2-[2-[2-[2-[[3-[[2-[1-[[[(S)-2-[4-(2,6-dichloro-benzoylamino)phenyl]-1-(methoxycarbonyl)ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium bromide

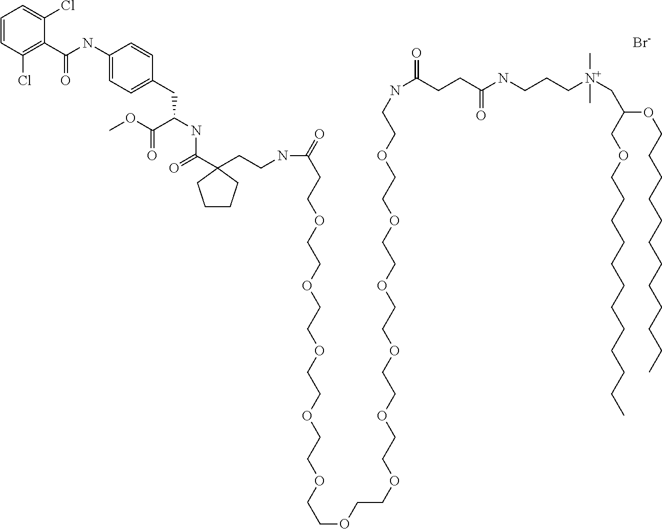 [SMILES: CCCCCCCCCCCCOCC(C[N+](C)(C)CCCNC(=O)CCC(=O)NCCOCCOCCOCCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)OC)CCCC1)OCCCCCCCCCCCC ; [Br-]]

To a mixture of 4-[[2-[2-[2-[2-[2-[2-[2-[2-[2-[2-[2-[2-[[3-[[2-[1-[[[(S)-1-(methoxycarbonyl)-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-4-oxobutanoic acid (0.052 mmol, 63 mg), N-hydroxysulfosuccinimide sodium salt (0.075 mmol, 16.5 mg), and 1-(3-dimethylaminopropyl)-3-ethylcarbodimide hydrochloride (0.063 mmol, 12 mg) in DMSO (2 mL) was added an excess DIPEA (0.26 mmol, 34 mg) at RT under nitrogen atmosphere. In 10 minutes, it gave a clear solution and it was stirred for 15 h at which time LCMS analysis indicated the formation of sulfo NHS ester intermediate. Then, the solid N-(3-aminopropyl)-2,3-bis(do decyloxy)-N,N-dimethylpropan-1-aminium bromide (0.032 mmol, 16 mg) was added at RT. The resulting solution was stirred for 2 h at which time high mass LCMS analysis indicated the presence of the desired product. Then, the excess DIPEA was removed under vacuum and the desired product was isolated by purification using HPLC method to obtain 34 mg (38% yield) of [2,3-bis(dodecyloxy)propyl][3-[[4-[[2-[2-[2-[2-[2-[2-[2-[2-[2-[2-[2-[2-[[3-[[2-[1-[[[(S)-2-[4-(2,6-dichloro-benzoylamino)phenyl]-1-(methoxycarbonyl)ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium bromide as a viscous oil. ES(+)-LRMS m/e calcd. for C₈₈H₁₅₃Cl₂N₆O₂₁ (M+)⁺ 1702.1342, obsd. 1702.3.

### b) Preparation of [2,3-bis(dodecyloxy)propyl][3-[[4-[[2-[2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1-4-dioxobutyl]amino]propyl]dimethylaminium trifluoroacetate

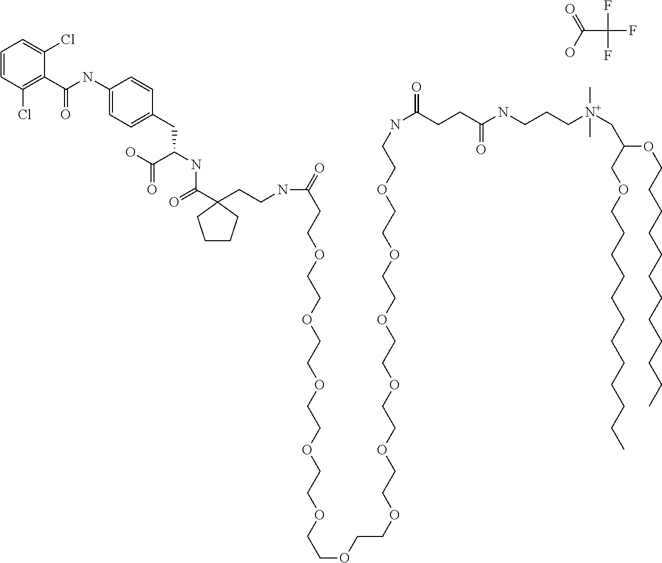 [SMILES: CCCCCCCCCCCCOCC(C[N+](C)(C)CCCNC(=O)CCC(=O)NCCOCCOCCOCCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCC ; O=C(O)C(F)(F)F]

To a solution of [2,3-bis(dodecyloxy)propyl][3-[[4-[[2-[2-[2-[2-[2-[2-[2-[2-[2-[2-[2-[2-[[3-[[2-[1-[[[(S)-2-[4-(2,6-dichloro-benzoylamino)phenyl]-1-(methoxycarbonyl)ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethyl-aminium bromide (0.018 mmol, 32 mg) in THF (4 mL) was added a warm aqueous solution of lithium hydroxide monohydrate (0.376 mmol, 9 mg) in water (1.0 mL) at RT. The resulting solution was stirred for 4 h at which time high mass LCMS analysis indicated the presence of the desired peak. Then, the THF was removed under vacuum and the desired product was isolated by purification using HPLC method to obtain 17 mg (54% yield) of [2,3-bis(dodecyloxy)propyl][3-[[44 [2-[2-[2-[2-[2-[2-[2-[2-[2-[2-[2-[2-[[3-[[2-[1-[[[(S)-1-carboxy-2-[4-(2,6-dichloro-benzoyl-amino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1-4-dioxobutyl]amino]propyl]dimethylaminium trifluoroacetate as a light yellow paste. ES(+)-HRMS m/e calcd. for C₈₇H₁₅₁Cl₂N₆O₂₁ (M)¹⁺ 1686.0304, obsd. 1686.0299.

### Preparation of (rac)-N-(3-aminopropyl)-2,3-bis-(hexadecyloxy)-N,N-dimethyl-1-propanaminium bromide

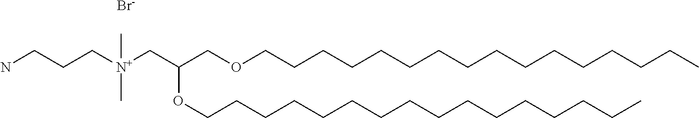 [SMILES: [Br-] ; CCCCCCCCCCCCCCCCOCC(C[N+](C)(C)CCCN)OCCCCCCCCCCCCCCCC]

### a) Preparation of Methanesulfonic Acid Hexadecyl Ester

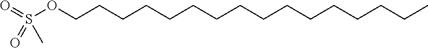 [SMILES: CCCCCCCCCCCCCCCCOS(C)(=O)=O]

A similar procedure as described in General method 1 and section a was used, starting from hexadecan-1-ol (200 mmol, 48.48 g), triethylamine (460 mmol, 64.1 mL), and methane-sulfonyl chloride (240 mmol, 27.5 g) to afford 60.5 g (94% yield) of methanesulfonic acid hexadecyl ester as an off-white solid. ES(+)-LRMS m/e calcd. for C₁₇H₃₆O₃S (M+H)⁺ 320.2385, obsd. 321.3.

### b) Preparation of (rac)-(2,3-bis(hexadecyloxy-propyl)-N,N-dimethyl-amine

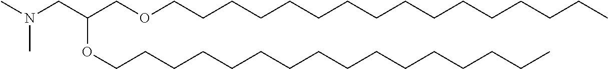 [SMILES: CCCCCCCCCCCCCCCCOCC(CN(C)C)OCCCCCCCCCCCCCCCC]

A similar procedure as described in General method 1 and section b was used, starting from sodium hydride (38.6 mmol, 1.54 g, 60% in oil), 3-(dimethylamino)-propane-1,2-diol (14.9 mmol, 1.77 g), and methanesulfonic acid hexadecyl ester (38.6 mmol, 12.4 g) to obtain 5.93 g (70% yield) of (rac)-(2,3-bis(hexadecyloxy-propyl)-N,N-dimethyl-amine as a colorless viscous oil. ES(+)-LRMS m/e calcd. for C₃₇H₇₇NO₂ (M+H)⁺ 567.5954, obsd. 568.6.

### c) Preparation of (rac)-N-[2,3-bis(hexadecyloxy)propyl]-1,3-dihydro-N,N-dimethyl-1,3-dioxo-2H-isoindole-2-propanaminium bromide

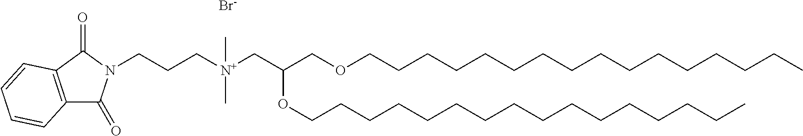 [SMILES: [Br-] ; CCCCCCCCCCCCCCCCOCC(C[N+](C)(C)CCCN1C(=O)c2ccccc2C1=O)OCCCCCCCCCCCCCCCC]

A similar procedure as described in General method 1 and section c was used, starting from 2-(3-bromopropyl)-isoindoline-1,3-dione (19.5 mmol, 5.23 g) and (rac)-(2,3-bis(hexa-decyloxy-propyl)-N,N-dimethyl-amine (10.4 mmol, 5.92 g) to prepare 7.57 g (96% yield) of (rac)-N-[2,3-bis(hexadecyloxy)propyl]-1,3-dihydro-N,N-dimethyl-1,3-dioxo-2H-isoindole-2-propanaminium bromide as a off-white paste. ES(+)-LRMS m/e calcd. for C₄₈H₈₇N₂O₄ (M+H)⁺ 755.67, obsd. 755.8.

### d) Preparation of (rac)-N-(3-aminopropyl)-2,3-bis(hexadecyloxy)-N,N-dimethyl-1-propanaminium bromide

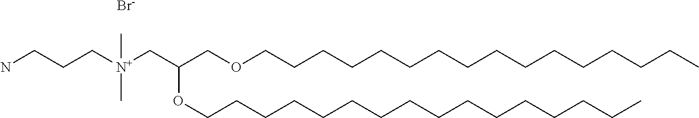 [SMILES: [Br-] ; CCCCCCCCCCCCCCCCOCC(C[N+](C)(C)CCCN)OCCCCCCCCCCCCCCCC]

A similar procedure as described in General method 1 and section d was used, starting from (rac)-N-[2,3-bis(hexade cyloxy)propyl]-1,3-dihydro-N,N-dimethyl-1,3-dioxo-2H-isoindole-2-propanaminium bromide (5.91 mmol, 4.47 g) and anhydrous hydrazine (201 mmol, 6.44) to give 1.5 g (40% yield) of a mixture of (rac)-N-(3-aminopropyl)-2,3-bis(hexadecyloxy)-N,N-dimethyl-1-propanaminium bromide and (rac)-N-(2,3-bis(hexadecyloxy propyl)-N-methyl-propan-1,3-diamine as a white solid. ES(+)-LRMS m/e for (rac)-N-(3-aminopropyl)-2,3-bis(hexadecyloxy)-N,N-dimethyl-1-propanaminium bromide calcd. for C₄₀H₈₅N₂O₂ (M+H)⁺ 625.6611, obsd. 625.9.

### Example 3

### Preparation of [[2,3-bis(hexadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate

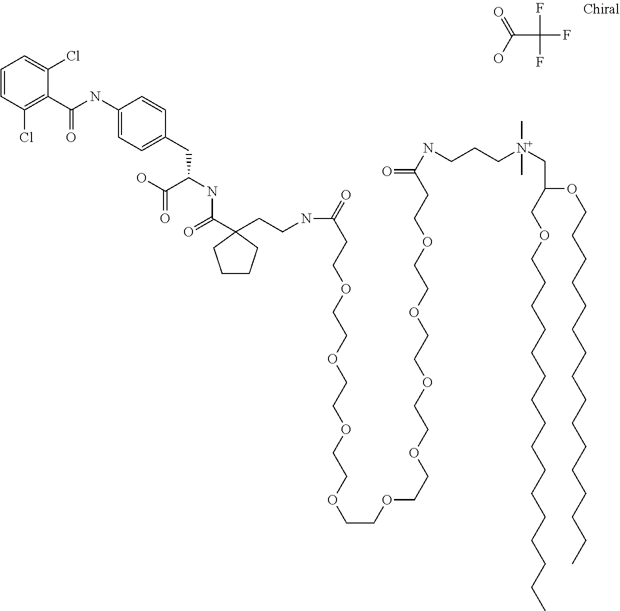 [SMILES: CCCCCCCCCCCCCCCCOCC(C[N+](C)(C)CCCNC(=O)CCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCCCCCC ; O=C(O)C(F)(F)F]

A similar procedure as described in General method 2 was used, starting from bis-(2,5-dioxopyrrolidin-1-yl)-4,7,10,13,16,19,22,25,28-nonaoxahentriacontane-1,31-dioate (0.141 mmol, 100 mg), (S)-2-[[1-(2-amino-ethyl)-cyclopentanecarbonyl]-amino]-3-[4-(2,6-dichloro-benzoyl-amino)-phenyl]-propionic acid trifluoroacetate salt (0.141 mmol, 69.5 mg), N-(3-aminopropyl)-2,3-bis(hexadecyloxy)-N,N-dimethylpropan-1-aminium bromide (0.169 mmol, 87 mg), and DIPEA (0.847 mmol, 109 mg) to afford after HPLC purification 100 mg (44% yield) of [[2,3-bis(hexadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclo-pentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate as a white solid. ES(+)-HRMS m/e calcd. for C₈₆H₁₅₀Cl₂N₅O₁₇ (M+)⁺ 1595.0399, obsd. 1595.0387.

### Example 4

### Preparation of [2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxyl ethoxy]ethoxy]ethoxy]ethoxy]ethyl]amino]-1,4-dioxobutyl]amino]propyl]dimethylaminium trifluoroacetate

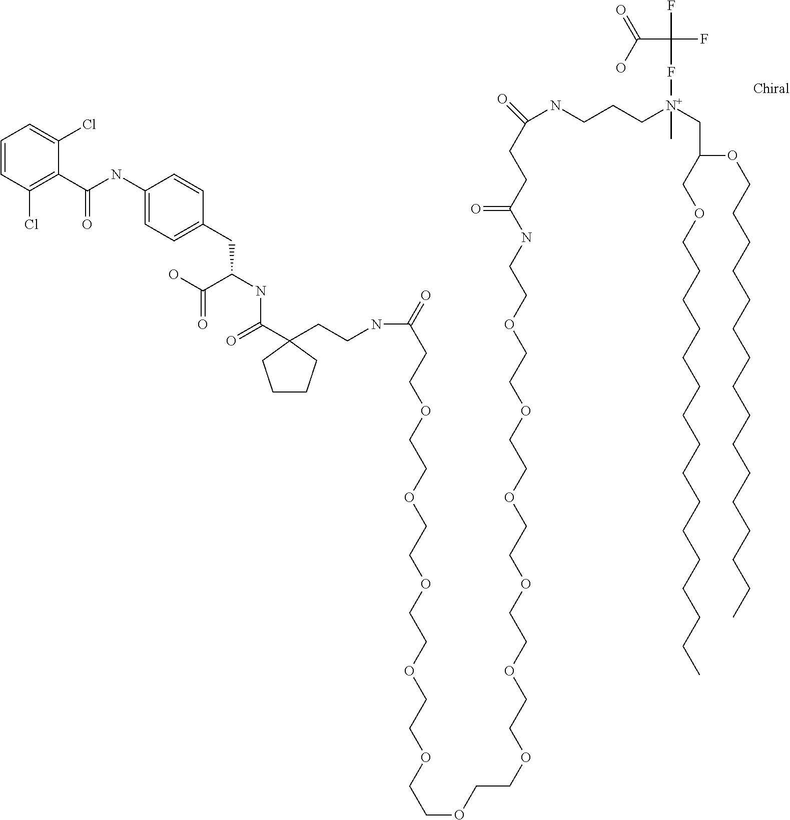

### a) Preparation of [2,3-bis(hexadecyloxy)propyl][3-[[4-[[2-[2-[2-[2-[2-[2-[2-[2-[2-[2-[2-[2-[[3-[[2-[1-[[[(S)-1-carboxymethyl-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium bromide and [2,3-bis(hexadecyloxy)propyl][3-[[4-[[2-[2-[2-[2-[2-[2-[2-[2-[2-[2-[2-[2-[[3-[[2-[1-[[[(S)-1-carboxymethyl-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]methylammonium

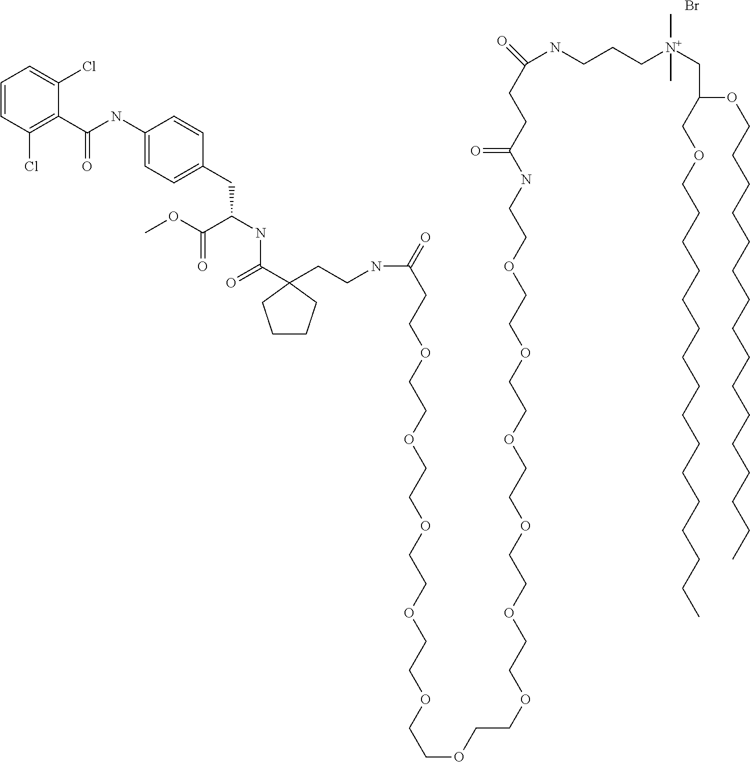 [SMILES: Br ; CCCCCCCCCCCCCCCCOCC(C[N+](C)(C)CCCNC(=O)CCC(=O)NCCOCCOCCOCCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)OC)CCCC1)OCCCCCCCCCCCCCCCC]

A similar procedure as described in General method 3 section a was used, starting from of [3-[[3-[2-[2-[2-[2-[2-[2-[2-[2-[2-[2-[2-[[3-[[2-[1-[[[(S)-1-carboxymethyl-2-[4-(2,6-dichloro-benzoyl-amino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-succinic acid (0.39 mmol, 475 mg), N-hydroxysulfosuccinimide sodium salt (0.57 mmol, 123 mg), 1-(3-dimethylaminopropyl)-3-ethylcarbodimide hydrochloride (0.47 mmol, 91 mg), a mixture of (rac)-N-(aminopropyl)-N,N-dimethyl-2,3-bis(hexade cyl-oxy)-1-propanaminium bromide and (rac)-N-(2,3-bis(hexadecyloxy propyl)-N-methyl-propan-1,3-diamine (0.47 mmol, 296 mg), and DIPEA (1.97 mmol, 254 mg) to obtain, after SFC separation, 79 mg (11% yield) of [2,3-bis(hexadecyloxy)propyl][3-[[4-[[2-[2-[2-[2-[2-[2-[2-[2-[2-[2-[2-[2-[[3-[[2-[1-[[[(S)-1-carboxy-methyl-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium bromide as a viscous oil, ES(+)-HRMS m/e calcd. for C₉₆H₁₆₉Cl₂N₆O₂₁ (M+H)²⁺ 906.5893, obsd. 906.5900, and 165 mg (23% yield) of [2,3-bis(hexadecyloxy)propyl][3-[[4-[[2-[2-[2-[2-[2-[2-[2-[2-[2-[2-[2-[2-[[3-[[2-[1-[[[(S)-1-carboxymethyl-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]methylammonium as a white solid, ES(+)-HRMS m/e calcd. for C₉₅H₁₆₆Cl₂N₆O₂₁ (M+2H)² 899.5814, obsd. 899.5818.

### b) Preparation of [2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl dimethyl-aminium trifluoroacetate

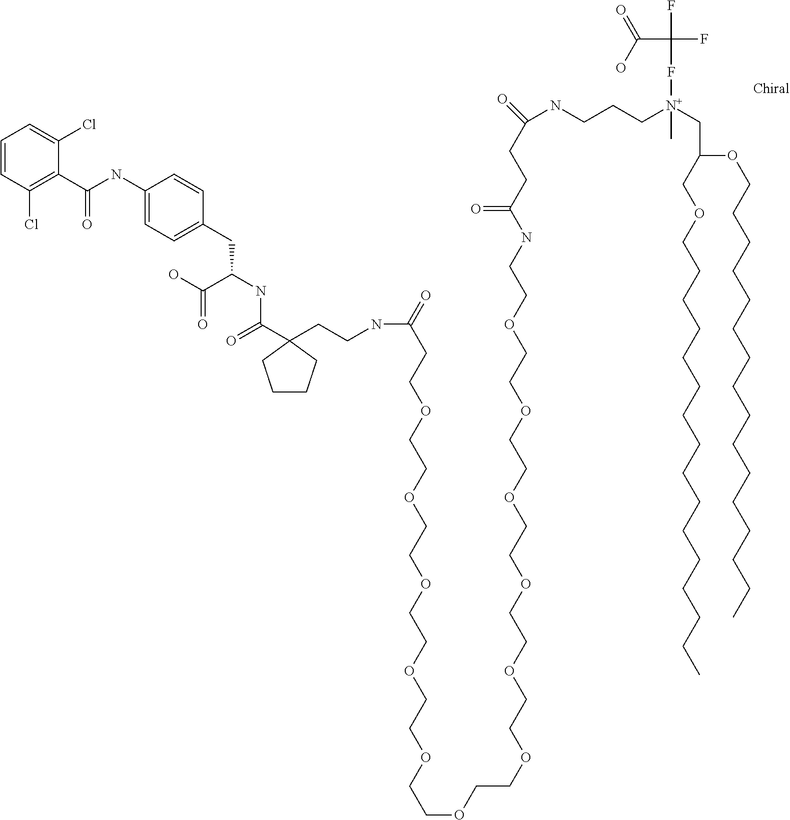

A similar procedure as described in General method 3 section b was used, starting from [2,3-bis(hexadecyloxy)propyl][3-[[4-[[2-[2-[2-[2-[2-[2-[2-[2-[2-[2-[2-[2-[[3-[[2-[1-[[[(S)-1-carboxy methyl-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium bromide (0.04 mmol, 75 mg) and lithium hydroxide monohydrate (0.8 mmol, 20 mg) to obtain 70 mg (75% yield) of after purification by HPLC [2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichlorobenzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium trifluoroacetate as a white solid paste. ES(+)-HRMS m/e calcd. for C₉₅H₁₆₇Cl₂N₆O₂₁ (M+H)²⁺ 899.5814, obsd. 899.5812.

### Example 5

### Preparation of (S)-Alpha-[[[1-[2-[[3-[2-[2-[2-[2-[2-[2-[2-[2-[2-[2-[2-[2-[[4-[[3-[[2,3-bis(hexadecyloxy)propyl]methylamino]propyl]amino]-1,4-dioxobutyl]amino]ethoxy]ethoxy]ethoxy]ethoxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino) benzenepropanoic acid

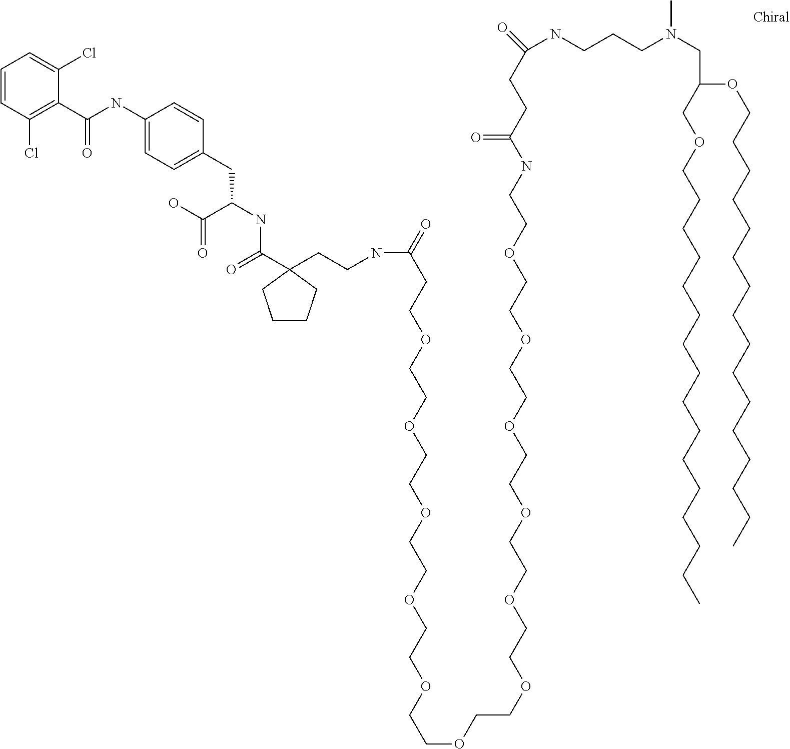 [SMILES: CCCCCCCCCCCCCCCCOCC(CN(C)CCCNC(=O)CCC(=O)NCCOCCOCCOCCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCCCCCC]

A similar procedure as described in General method 3 section b was used, starting from [2,3-bis(hexadecyloxy)propyl][3-[[4-[[2-[2-[2-[2-[2-[2-[2-[2-[2-[2-[2-[2-[[3-[124]-[[[(S)-1-carboxy-methyl-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]methylammonium (0.09 mmol, 162 mg) and lithium hydroxide monohydrate (1.8 mmol, 43 mg) to obtain 120 mg (75% yield) of (S)-alpha-[[[1-[2-[[3-[2-[2-[2-[2-[2-[2-[2-[2-[2-[2-[2-[2-[[4-[[3-[[2,3-bis(hexadecyloxy)propyl]methylamino]propyl]amino]-1,4-dioxobutyl]amino]ethoxy]ethoxy]ethoxy]ethoxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino)benzenepropanoic acid as a white solid. ES(+)-HRMS m/e calcd. for C₉₄H₁₆₄Cl₂N₆O₂₁ (M+2H)² 892.5736, obsd. 892.5743.

### Preparation of (rac)-N-(3-aminopropyl)-N,N-dimethyl-2,3-bis(octadecyloxy)-1-propanaminium bromide

### a) Preparation of Methanesulfonic Acid Octadecyl Ester

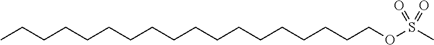 [SMILES: CCCCCCCCCCCCCCCCCCOS(C)(=O)=O]

A similar procedure as described in General method 1 and section a was used, starting from octadecan-1-ol (100 mmol, 27.05 g), triethylamine (230 mmol, 32.1 mL), and methane-sulfonyl chloride (120 mmol, 13.7 g) to afford 29.71 g (85% yield) of methanesulfonic acid octadecyl ester as a light yellow solid. ES(+)-LRMS m/e calcd. for C₁₉H₄₀O₃S (M+H)⁺ 349.2698, obsd. 349.2.

### b) Preparation of (rac)-(2,3-bis(octadecyloxy-propyl)-N,N-dimethyl-amine

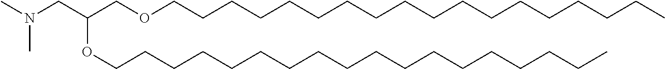 [SMILES: CCCCCCCCCCCCCCCCCCOCC(CN(C)C)OCCCCCCCCCCCCCCCCCC]

A similar procedure as described in General method 1 and section b was used, starting from sodium hydride (38.6 mmol, 1.54 g, 60% in oil), 3-(dimethylamino)-propane-1,2-diol (14.9 mmol, 1.77 g), and methanesulfonic acid octadecyl ester (38.6 mmol, 13.5 g) to obtain 4.4 g (47% yield) of (rac)-(2,3-bis(octadecyloxy-propyl)-N,N-dimethyl-amine as a white. ES(+)-LRMS m/e calcd. for C₄₁11₈₅NO₂ (M+H)⁺ 624.6580, obsd. 624.8.

### c) Preparation of (rac)-N-[2,3-bis(octadecyloxy)propyl]-1,3-dihyro-N,N-dimethyl-1,3-dioxo-2H-isoindole-2-propanaminium bromide

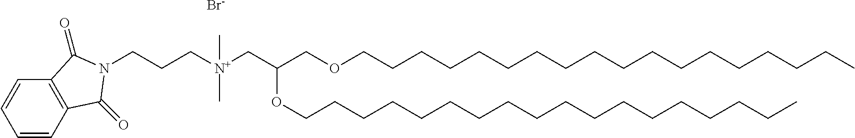 [SMILES: [Br-] ; CCCCCCCCCCCCCCCCCCOCC(C[N+](C)(C)CCCN1C(=O)c2ccccc2C1=O)OCCCCCCCCCCCCCCCCCC]

A similar procedure as described in General method 1 and section c was used, starting from 2-(3-bromopropyl)-isoindoline-1,3-dione (11.2 mmol, 3.01 g) and (rac)-(2,3-bis(octadecyl-oxy-propyl)-N,N-dimethyl-amine (5.61 mmol, 3.5 g) to prepare 4.03 g (88% yield) of (rac)-N-[2,3-bis(octadecyloxy)propyl]-1,3-dihyro-N,N-dimethyl-1,3-dioxo-2H-isoindole-2-propanaminium bromide as a light brown solid. ES(+)-LRMS m/e calcd. for C₅₂H₉₅N₂O₄ (M+H)⁺ 811.7292, obsd. 811.8.

### d) Preparation of (rac)-N-(3-aminopropyl)-N,N-dimethyl-2,3-bis(octadecyloxy)-1-propanaminium bromide

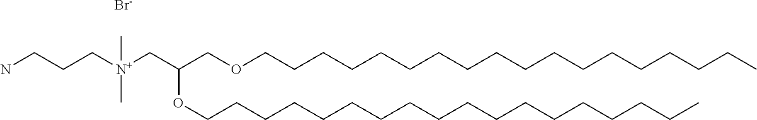 [SMILES: [Br-] ; CCCCCCCCCCCCCCCCCCOCC(C[N+](C)(C)CCCN)OCCCCCCCCCCCCCCCCCC]

A similar procedure as described in General method 1 and section d was used, starting from (rac)-N-[2,3-bis(octadecyloxy)propyl]-1,3-dihyro-N,N-dimethyl-1,3-dioxo-2H-isoindole-2-propanaminium bromide (4.96 mmol, 4.03 g) and anhydrous hydrazine (198 mmol, 6.36) to give 0.4 g (12% yield) of (rac)-N-(3-aminopropyl)-N,N-dimethyl-2,3-bis(octadecyloxy)-1-propanaminium bromide as a off-white solid. ES(+)-LRMS m/e calcd. for C₄₄H₉₃N₂O₂ (M+H)⁺ 682.22, obsd. 682.1.

### Example 6

### Preparation of [2,3-bis(octadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide

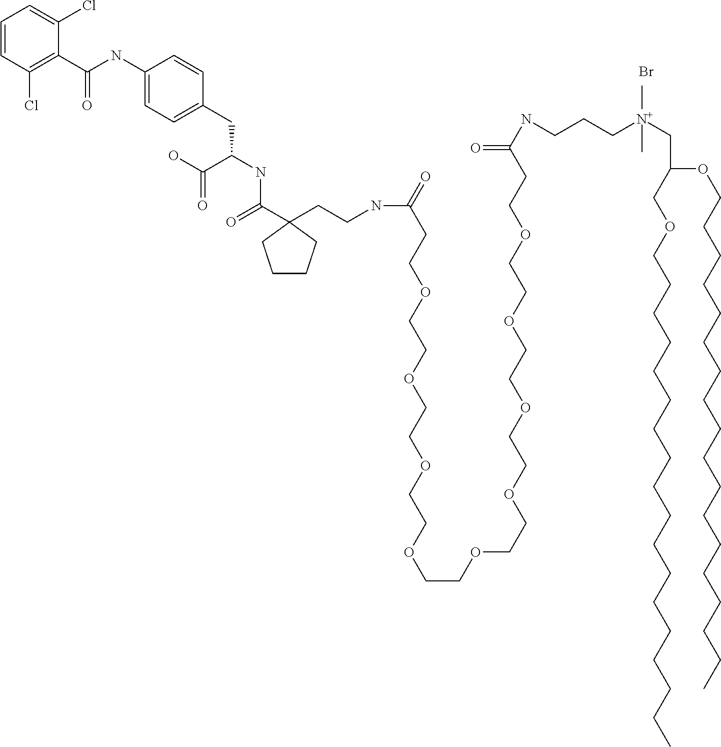 [SMILES: CCCCCCCCCCCCCCCCCCOCC(C[N+](C)(C)CCCNC(=O)CCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCCCCCCCC ; Br]

A similar procedure as described in General method 2 was used, starting from bis-(2,5-dioxopyrrolidin-1-yl)-4,7,10,13,16,19,22,25,28-nonaoxahentriacontane-1,3′-dioate (0.3 mmol, 220 mg), (S)-2-[[1-(2-amino-ethyl)-cyclop entanecarbonyl]-amino]-3-[4-(2,6-dichloro-benzoyl-amino)-phenyl]-propionic acid trifluoroacetate salt (0.3 mmol, 153 mg), N-(3-aminopropyl)-2,3-bis(octadecyloxy)-N,N-dimethylpropan-1-aminium bromide (0.37 mmol, 254 mg), and DIPEA (1.86 mmol, 241 mg) to obtain after crystallizaton 218 mg (42% yield) of [2,3-bis(octadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide salt as a white solid. ES(+)-LRMS m/e calcd. for C₉₀H₁₅₈Cl₂N₅O₁₇ (M+)⁺1651.1030, obsd. 1651.1.

### Preparation of (rac)-N-(3-aminopropyl)-2,3-bis-(icosyloxy)-N,N-dimethyl-1-propanaminium bromide

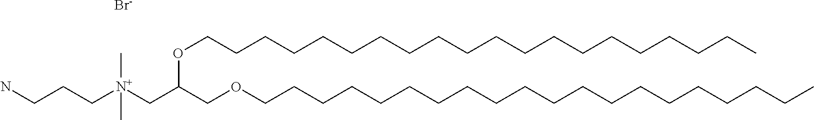 [SMILES: [Br-] ; CCCCCCCCCCCCCCCCCCCCOCC(C[N+](C)(C)CCCN)OCCCCCCCCCCCCCCCCCCCC]

### a) Preparation of Methanesulfonic Acid Icosyl Ester

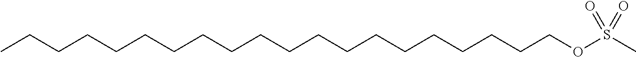 [SMILES: CCCCCCCCCCCCCCCCCCCCOS(C)(=O)=O]

A similar procedure as described in General method 1 and section a was used, starting from icosan-1-ol (33.5 mmol, 10 g), triethylamine (77 mmol, 10.7 mL), and methanesulfonyl chloride

(40.2 mmol, 4.6 g) to afford 9.78 g (77% yield) of methanesulfonic acid icosyl ester as a white solid. ES(+)-LRMS m/e calcd. for C₂₁H₄₄O₃S (M+H)⁺ 377.3, obsd. 377.3.

### b) Preparation of (rac)-(2,3-bis(icosyloxy-propyl)-N,N-dimethyl-amine

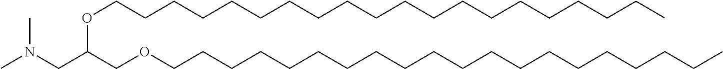 [SMILES: CCCCCCCCCCCCCCCCCCCCOCC(CN(C)C)OCCCCCCCCCCCCCCCCCCCC]

A similar procedure as described in General method 1 and section b was used, starting from sodium hydride (26 mmol, 1.2 g, 60% in oil), 3-(dimethylamino)-propane-1,2-diol (9.99 mmol, 1.19 g), and methanesulfonic acid icosyl ester (26 mmol, 9.78 g) to obtain 3.63 g (53% yield) of (rac)-(2,3-bis(icosyloxy-propyl)-N,N-dimethyl-amine as a white solid. ES(+)-LRMS m/e calcd. for C₄₅H₉₃NO₂ (M+H)⁺ 680.23, obsd. 680.9.

### c) Preparation of (rac)-N-[2,3-bis(icosyloxy)propyl]-1,3-dihyro-N,N-dimethyl-1,3-dioxo-2H-isoindole-2-propanaminium bromide

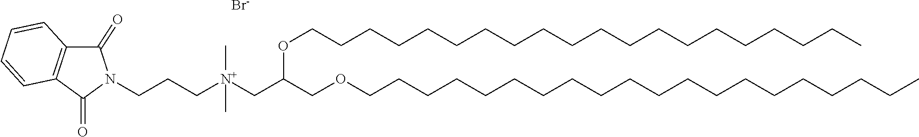 [SMILES: [Br-] ; CCCCCCCCCCCCCCCCCCCCOCC(C[N+](C)(C)CCCN1C(=O)c2ccccc2C1=O)OCCCCCCCCCCCCCCCCCCCC]

A similar procedure as described in General method 1 and section c was used, starting from 2-(3-bromopropyl)-isoindoline-1,3-dione (9.98 mmol, 2.68 g) and (rac)-(2,3-bis(icosyloxy-propyl)-N,N-dimethyl-amine (5.34 mmol, 3.63 g) to prepare 4.57 g (98% yield) of (rac)-N-[2,3-bis(icosyloxy)propyl]-1,3-dihyro-N,N-dimethyl-1,3-dioxo-2H-isoindole-2-propan-aminium bromide as a light brown solid. ES(+)-LRMS m/e calcd. for C₅₆H₁₀₃N₂O₄ (M+H)⁺ 868.43, obsd. 868.0.

### d) Preparation of (rac)-N-(3-aminopropyl)-2,3-bis-(icosyloxy)-N,N-dimethyl-1-propanaminium bromide

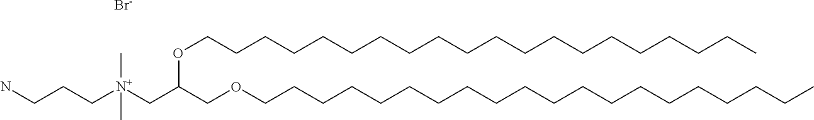 [SMILES: [Br-] ; CCCCCCCCCCCCCCCCCCCCOCC(C[N+](C)(C)CCCN)OCCCCCCCCCCCCCCCCCCCC]

A similar procedure as described in General method 1 and section d was used, starting from (rac)-N,N-dimethyl-N-(3-phthalimido)-propyl-2,3-bis(icosyloxy)-1-propaniminium bromide (5.26 mmol, 4.57 g) and anhydrous hydrazine (179 mmol, 5.73) to give 0.3 g (8% yield) of (rac)-N-(3-aminopropyl)-2,3-bis-(icosyloxy)-N,N-dimethyl-1-propanaminium bromide as an off-white solid, ES(+)-LRMS m/e calcd. for C₄₈H₁₀₁N₂O₂ (M+H)⁺ 738.33, obsd. 738.0, and (rac)-N-(2,3-bis(icosyloxy propyl)-N-methyl-propan-1,3-diamine as a white solid. ES(+)-LRMS m/e calcd. for C₄₇H₉₈N₂O₂ (M+H)⁺ 723.9, obsd. 723.8.

### Example 7

### Preparation of [2,3-bis(icosanyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide

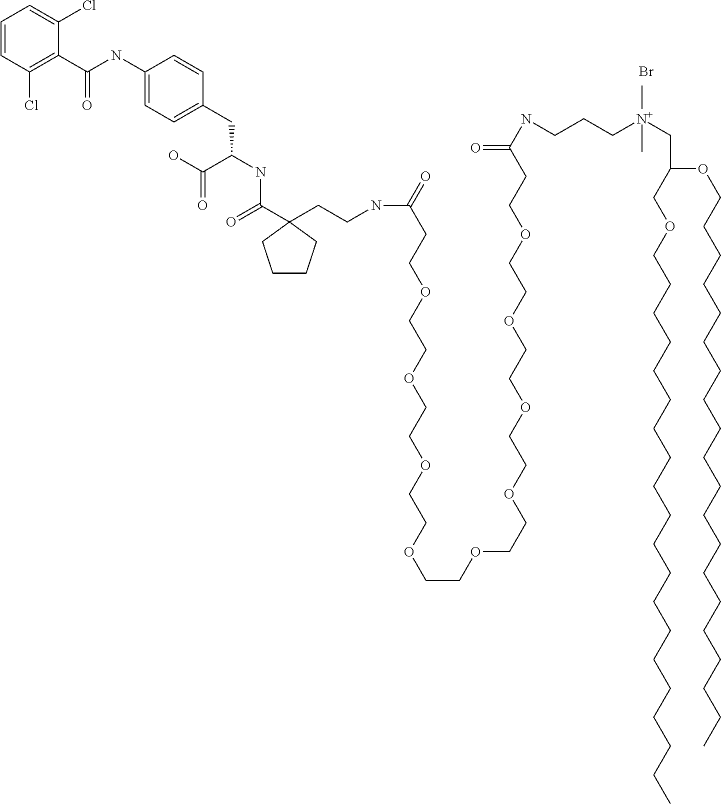 [SMILES: CCCCCCCCCCCCCCCCCCCCOCC(C[N+](C)(C)CCCNC(=O)CCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCCCCCCCCCC ; Br]

A similar procedure as described in General method 2 was used, starting from bis-(2,5-dioxopyrrolidin-1-yl)-4,7,10,13,16,19,22,25,28-nonaoxahentriacontane-1,3′-dioate (0.21 mmol, 150 mg), (S)-2-[[1-(2-amino-ethyl)-cyclop entanecarbonyl]-amino]-3-[4-(2,6-dichloro-benzoyl-amino)-phenyl]-propionic acid trifluoroacetate salt (0.21 mmol, 104 mg), (rac)-N-(3-aminopropyl)-2,3-bis-(icosyloxy)-N,N-dimethyl-1-propanaminium bromide (0.25 mmol, 188 mg), and DIPEA (1.27 mmol, 164 mg) to obtain after crystallization 130 mg (36% yield) of [2,3-bis(icosanyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide salt as a white solid. ES(+)-LRMS m/e calcd. for C₉₄H₁₆₆Cl₂N₅O₁₇ (M+)⁺ 1709.25, obsd. 1709.4.

### Example 8

### Preparation of (S)-Alpha-[[[1-[2-[[3-[2-[2-[2-[2-[2-[2-[2-[2-[[3-[[3-[[2,3-bis(icosanyloxy)propyl]methylamino]propyl]amino]-3-oxopropyl]oxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino)benzenepropanoic acid

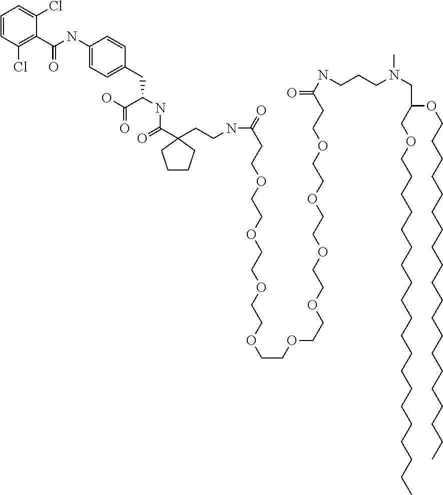 [SMILES: CCCCCCCCCCCCCCCCCCCCOCC(CN(C)CCCNC(=O)CCOCCOCCOCCOCCOCCOCCOCCOCCOCCC(=O)NCCC1(C(=O)N[C@@H](Cc2ccc(NC(=O)c3c(Cl)cccc3Cl)cc2)C(=O)O)CCCC1)OCCCCCCCCCCCCCCCCCCCC]

A similar procedure as described in General method 2 was used, starting from bis-(2,5-dioxopyrrolidin-1-yl)-4,7,10,13,16,19,22,25,28-nonaoxahentriacontane-1,3′-dioate (0.17 mmol, 120 mg), (S)-2-[[1-(2-amino-ethyl)-cyclopentanecarbonyl]-amino]-3-[4-(2,6-dichloro-benzoylamino)-phenyl]-propionic acid trifluoroacetate salt (0.17 mmol, 83.4 mg), (rac)-N-(2,3-bis(icosyloxy propyl)-N-methyl-propan-1,3-diamine (0.21 mmol, 147 mg), and DIPEA (1.27 mmol, 131 mg) to obtain 113 mg (39% yield) of (S)-Alpha-[[[1-[2-[[3-[2-[2-[2-[2-[2-[2-[2-[2-[3-[[3-[[3-[[2,3-bis(ico s anyloxy)propyl]methylamino]propyl]amino]-3-oxo-propyl]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino)benzenepropanoic acid as a white solid. ES(+)-LRMS m/e calcd. for C₉₃H₁₆₃Cl₂N₅O₁₇ (M+)⁺ 1695.23, obsd. 1695.4.

### Example 9

### Preparation of (rac)-N-(3-amino-propyl)-(2,3-bis-octadecyloxy-propyl)-N,N-dimethyl-ammonium bromide

### a) Preparation of Methanesulfonic Acid Octadecyl Ester

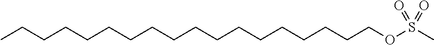 [SMILES: CCCCCCCCCCCCCCCCCCOS(C)(=O)=O]

Octadecan-1-ol (27.05 g, 100 mmol) and methanesulfonyl chloride (13.2 g, 8.94 ml, 115 mmol) were combined in anhydrous THF (200 ml) and cooled in ice/water bath. Then triethylamine (13.2 g, 18.1 ml, 130 mmol) in 40 mL anhydrous THF was added dropwise and after the addition was completed, the cooling bath was removed. The reaction mixture was stirred at RT for 30 min and then extracted with 2 N hydrochloric acid (200 mL). The organic layer was separated, dried over anhydrous sodium sulfate and concentrated under reduced pressure to afford white solid, which was recrystallized from hot hexane. 24.9 g of white needle-like crystals were isolated. The mother liquor was concentrated, white solid fell out and additional 7.2 g of amorphous white material was isolated by filtration. ES(+)-LRMS m/e calcd. for C₁₉H₄₀O₃SNa (M+Na)⁺ 371.3, obsd. 371.2.

### b) Preparation of (rac)-2,3-bis(octadecyloxy-propyl)-N,N-dimethyl-amine

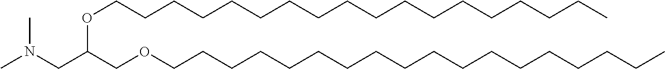 [SMILES: CCCCCCCCCCCCCCCCCCOCC(CN(C)C)OCCCCCCCCCCCCCCCCCC]

Sodium hydride (2.01 g, 50.4 mmol, 60% in oil) was suspended in THF (20 ml) and 3-(dimethylamino)propane-1,2-diol (1.5 g, 12.6 mmol) in THF (10 mL) was added dropwise. The resulting solution was stirred for 10 min at RT and then a solution of methanesulfonic acid octadecyl ester (11.0 g, 31.5 mmol) in THF (40 mL) was added dropwise. The resulting reaction mixture was refluxed overnight. Then it was quenched with methanol and partitioned between diethyl ether and water. Organic layer was washed with brine and dried over anhydrous sodium sulfate. It was purified on silica gel by flash chromatography using methanol/methylene chloride. The title compound was isolated as yellowish oil which solidified upon standing (4.5 g). ES(+)-LRMS m/e calcd. for C₄₁H₈₅NO₂ (M+H)⁺ 624.7, obsd. 624.8.

### c) Preparation of (rac)-N-[2,3-bis(octadecyloxy)propyl]-1,3-dihyro-N,N-dimethyl-1,3-dioxo-2H-isoindole-2-propanaminium bromide

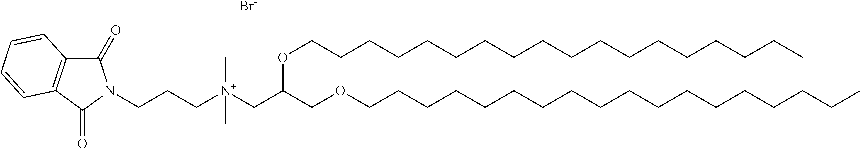 [SMILES: [Br-] ; CCCCCCCCCCCCCCCCCCOCC(C[N+](C)(C)CCCN1C(=O)c2ccccc2C1=O)OCCCCCCCCCCCCCCCCCC]

(rac)-2,3-Bis(octadecyloxy-propyl)-N,N-dimethyl-amine (1.107 g, 1.77 mmol) and 2-(3-bromopropyl)isoindoline-1,3-dione (499 mg, 1.86 mmol) were combined in DMF (5 ml) and stirred at 100° C. overnight. The reaction mixture almost solidified upon cooling to RT. It was then triturated with acetonitrile and the solid material was collected by filtration (1.1 g). Then it was crystallized from ethanol and 841 mg of title compound was isolated. ES(+)-LRMS m/e calcd. for C₅₂H₉₅N₂O₄, M+811.7, obsd. 811.8.

### d) Preparation of (rac)-N-(3-amino-propyl)-(2,3-bis-octadecyloxy-propyl)-N,N-dimethyl-ammonium bromide

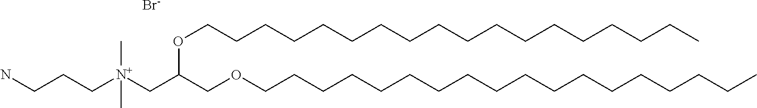 [SMILES: [Br-] ; CCCCCCCCCCCCCCCCCCOCC(C[N+](C)(C)CCCN)OCCCCCCCCCCCCCCCCCC]

To a solution of (rac)-N-(2,3-bis-octadecyloxy-propyl)₄₃-(1,3-dioxo-1,3-dihydro-isoindol-2-yl)-propyl]-N,N-dimethyl-ammonium bromide (200 mg, 224 μmol) in ethanol (2.5 mL) was added hydrazine (71.8 mg, 70.4 μl, 2.24 mmol). The reaction mixture was stirred at RT for 2 days. White precipitate appeared during the course of the reaction. The reaction mixture was then diluted with chloroform (15 mL) and the insoluble material was filtered out and discarded. Then the filtrate was concentrated under reduced pressure and upon trituration with acetonitrile a white solid precipitated and 40 mg of the title compound was isolated by filtration. The filtrate was left in a freezer overnight and a new batch of white solid was collected (additional 78 mg of title compound). ES(+)-LRMS m/e calcd. for C₄₄H₉₃N₂O₂, M+681.7, obsd. 681.6.

### Preparation of: [2,3-bis(octadecyloxy)propyl][3-[[3-[2-[2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethoxy]-1-oxopropyl]amino]propyl]dimethylaminium trifluoroacetate

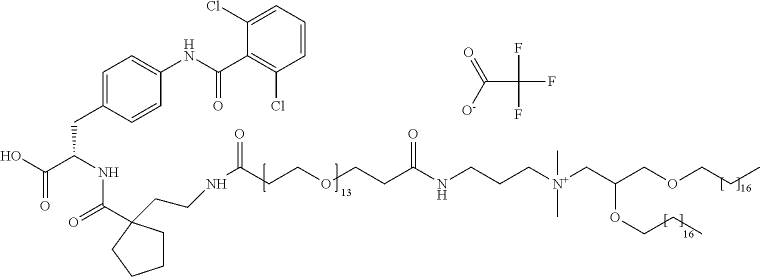

To a solution of (S)-2-(1-(2-aminoethyl)cyclopentanecarboxamido)-3-(4-(2,6-dichloro-benzamido)phenyl)propanoic acid (70 mg, 142 μmol) in DMSO (2 mL) was added dropwise a DMSO solution (2 mL) of bis(2,5-dioxopyrrolidin-1-yl)-4,7,10,13,16,19,22,25,28,31,34,37,40-tridecaoxatritetracontane-1,43-dioate (126 mg, 142 μmol), followed by addition of DIPEA (36.7 mg, 49.7 μL, 284 μmol) and the resulting reaction mixture was stirred at RT for 30 min. Then, a chloroform solution (3 mL) of (rac)-N-(3-amino-propyl)-(2,3-bis-octadecyloxy-propyl)-N,N-dimethyl-ammonium bromide (97.0 mg, 142 μmol) was added dropwise to the reaction mixture and the resulting mixture was stirred at RT overnight. Then, chloroform was removed under reduced presssure. Crude material was purified by reverse-phase HPLC and yield 72 mg of title compound after lyophilization. ES(+)-LRMS m/e calcd. for C₉₈H₁₇₄Cl₂N₅O₂₁, M+ 1827.2, obsd. 1827.2.

### Example 10

### Formation of VLA-4-Targeted Lipid Nanoparticles

Lipid nanoparticles comprising: (1) 2-(2,2-di((9Z,12Z)-octadeca-9,12-dienyl)-1,3-dioxolan-4-yl)-N,N-dimethyl-ethanamine; (2) a VLA-4-targeting lipid of the present invention, (3) phospholipid, (4) cholesterol, (5) hyaluronic acid (HA) and (6) siRNA; and variations thereof were formed using an ethanol injection method. Specifically, Table 5 (below) provides a description of the specific ingredients, molar ratios of each component, and the weight ratio of hyaluronic acid:siRNA for each lipid nanoparticle formulation that was synthesized. Table 6 provides a description of the diameter and the zeta potential (if measured) of each lipid nanoparticle formulation that was synthesized. The lipid portion of each formulation [(1)-(4) above] was dissolved in absolute ethanol (200 proof). In a separate vial (borosilicate, Schott Fiolax Clear), the hyaluronic acid portion (5) and siRNA (6) was diluted to an appropriate concentration (0.05-2 mg/mL for each hyaluronic acid and siRNA) using either nuclease-free water (Qiagen), citrate buffer (20 mM, pH 4.0), or 5% Dextrose (D5W). The lipid-ethanol solution was then added with rapid stirring to the aqueous siRNA containing solution resulting in a final ethanol concentration of 10-50%. After standing at RT for 3-24 h, under argon and shielded from the light, the formed lipid nanoparticle suspension was filtered using a 0.22 micron syringe filter (Millex-GV, Millipore) prior to use. The following are definitions for the abbreviations used in Table 5 for each formulation number (Form. #):
(1) Dlin-KC2-DMA=2-(2,2-di((9Z,12Z)-octadeca-9,12-dienyl)-1,3-dioxolan-4-yl)-N,N-dimethyl-ethanamine;
(2) For VLA-4-targeting lipids:
Compound-1 (of example 1)=[2,3-bis(dodecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate;
Compound-3 (of example 3)=[[2,3-bis(hexadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate;
Compound-4 (of example 4)=[2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium trifluoroacetate;
Compound-5 (of example 5)=(S)-Alpha-[[[1-[2-[[3-[2-[2-[2-[2-[2-[2-[2-[2-[2-[2-[2-[2-[[4-[[3-[[2,3-bis(hexa decyloxy)propyl]methylamino]propyl]amino]-1,4-dioxobutyl]amino]ethoxy]ethoxy]ethoxy]ethoxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichloro-benzoylamino) benzenepropanoic acid
(3) For the phospholipid:
DSPC=1,2-distearoyl-sn-glycero-3-phosphocholine;
(4) Cholesterol=cholesterol.

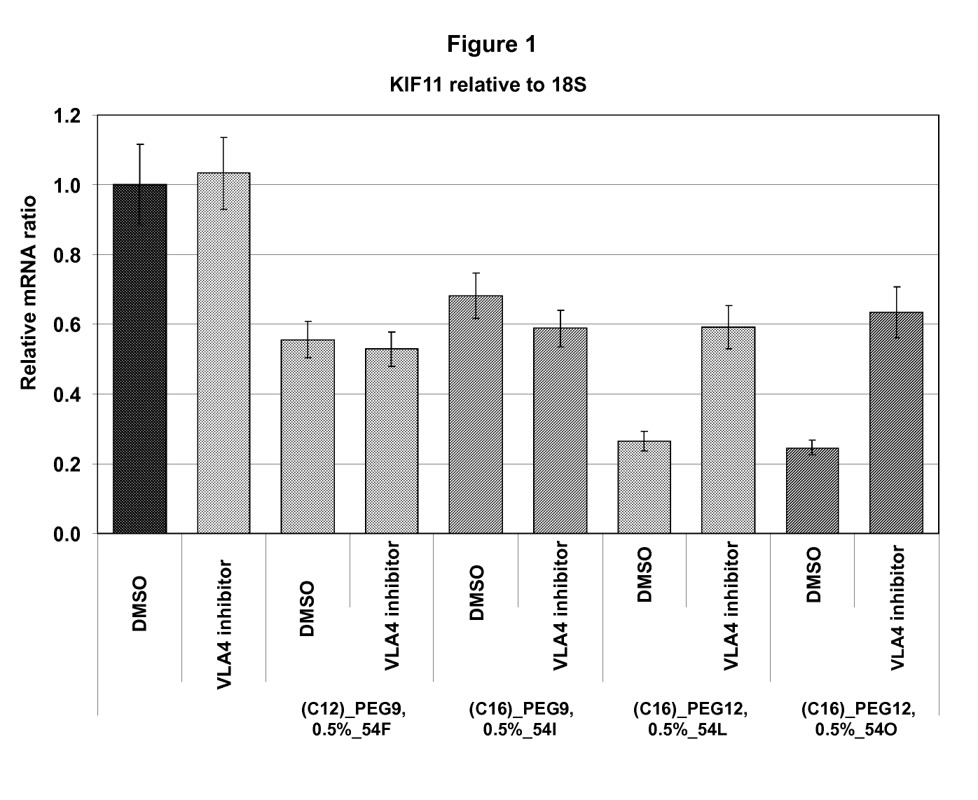

For example, in Table 5, the composition for formulation 54F:
Dlin-KC2-DMA/DSPC/Cholesterol/Compound 1,
   indicates that this lipid nanoparticle is comprised of:
(1) 2-(2,2-di((9Z,12Z)-octadeca-9,12-dienyl)-1,3-dioxolan-4-yl)-N,N-dimethyl-ethanamine, with a molar ratio of 40.6,
(2) [2,3-bis(dodecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate, with a molar ratio of 0.5,
(3) 1,2-distearoyl-sn-glycero-3-phosphocholine, with a molar ratio of 10.6,
(4) cholesterol, with a molar ratio of 48.7, and
(5 & 6) an equal mass of hyaluronic acid and siRNA and
   has an N/P ratio (defined as the molar ratio of cationic amines to nucleic acid phosphates) of 3.8, and (as shown in Table 6) has a particle diameter of 115.90 nm, a zeta potential of 0.17 meV, with results showing knockdown in FIG. 1. The siRNA used in these formulations was 21 nucleotides in length with 2 nucleotide overhangs at the 3′ end which target mRNA for KIF11. The siRNA sequence targeting the KIF11 sequence is comprised of ucGAGAAucuAAAcuAAcudTsdT 5′-3′ (SEQ ID 1) for the sense strand and AGUuAGUUuAGAUUCUCGAdTsdT 5′-3′ (SEQ ID 2) for the antisense strand (where lower case lettering=2′-OMe RNA modifications; upper case lettering=normal RNA; dT=deoxythymidine; and s=phosphorothioate).

TABLE 5
Compositions and molar ratios of lipid nanoparticles.
The weight ratio of hyaluronic acid to siRNA was
1.0 for each. The N/P ratio was 3.8 for each.

| Form. # | Composition | Molar Ratio |
|---|---|---|
| 54F | Dlin-KC2-DMA/DSPC/Cholesterol/ | 40.6:10.6:48.7:0.5 |
| | Compound-1 | |
| 54I | Dlin-KC2-DMA/DSPC/Cholesterol/ | 40.6:10.6:48.7:0.5 |
| | Compound-3 | |
| 54L | Dlin-KC2-DMA/DSPC/Cholesterol/ | 40.6:10.6:48.7:0.5 |
| | Compound-5 | |
| 54O | Dlin-KC2-DMA/DSPC/Cholesterol/ | 40.6:10.6:48.7:0.5 |
| | Compound-4 | |

TABLE 6
Diameter and the zeta potential (if
measured) of each lipid nanoparticle.

| | Particle Diameter | Zeta Potential |
|---|---|---|
| Formulation # | (nm) | (MeV) |
| 54F | 115.90 | 0.17 |
| 54I | 85.07 | −0.47 |
| 54L | 94.68 | −0.15 |
| 54O | 102.75 | −2.15 |

siRNA CONCENTRATION and % Encapsulation.

The ribogreen fluorescence assay was used to determine the concentration and percent entrapment of siRNA in each formulation preparation. Ribogreen dye fluoresces upon binding with double stranded nucleic acids and can thus be used to measure the amount of free siRNA in a sample. The amount of bound/encapsulated siRNA can be calculated after determining both the free/unbound siRNA and total siRNA concentrations. The total amount of siRNA can be obtained after disrupting the lipid nanoparticle and thus liberating the siRNA by using a surfactant such as Triton X-100. For this body of work, a commercially available ribogreen assay kit (Quanti-iT™ Ribogreen® RNA Assay Kit by Invitrogen) was used per manufacturer's instructions. siRNA sequences used in the preparation of the lipid nanoparticle (LNP) were used as standards. 1% Triton X-100 was used to disrupt the lipid nanoparticle suspension for the purpose of determining the total siRNA content. Ribogreen fluorescence was measured with a SpectraMax M5 (Molecular Devices) multi-mode microplate reader using excitation and emission wavelengths of 485 and 525 nm, respectively. The percent encapsulated siRNA was determined by using the following equation:

% Encapsulation=([siRNA]ₜₒₜₐₗ−[siRNA]_{free})/[siRNA]ₜₒₜₐₗ×100.

[siRNA]ₜₒₜₐₗ=the total concentration (mg/ml) of siRNA measured after disruption of the lipid nanoparticle. [siRNA]=the concentration (mg/ml) of free/unbound siRNA measured prior to the addition of Triton X-100. For all in vitro screening, the concentration of bound/encapsulated siRNA was used for dosing. For siRNA formulation nos. 54F, 54l, 54L, and 54O, an siRNA concentration of 0.038 mg/ml was calculated.

Particle Size and Zeta Potential.

Particle size and zeta potential were determined using a Malvern Zetasizer Nano-ZS Instrument. For particle size measurements, the lipid nanoparticles were diluted by a factor of 1 to 100 (20 μL diluted in 2000 μL buffer) using either phosphate buffered saline (PBS), 5% dextrose (D5W), or 20 mM citrate buffer (pH 4.0). Light scattering measurements were performed at 25° C. in polystyrene cuvettes. For zeta potential measurements, the lipid nanoparticles were diluted using nuclease free water using a dilution factor of 1 to 100 (20 μL diluted in 2000 μL of deionized water). The results are shown in Table 6.

### In Vitro Evaluation of Formulated Lipid Nanoparticle Activity

Cell Lines.

The human cancer cell line MV4-11 was maintained in media supplemented with 10% heat-inactivated Fetal Bovine Serum (HI-FBS; Gibco/BRL, Gaithersburg, Md.) and 2 mM L-glutamine (Gibco/BRL).

Transfection.

MV4-11 suspension cells in 1 ml of culturing medium were seeded in 24-well plates 24 h before transfection and RNA quantification targeting siRNA were formulated in LNPs and directly added to the medium for transfection. Cells were then collected for RNA quantification.

Sample collection and mRNA purification for in vitro studies were performed as follows. MV4-11 cells were lysed directly on the plate with RNA lysis buffer (Qiagen). Suspension cells were collected into tubes and spun down at 2,000 rpm for 1 min, then the cell pellets were lysed with RNA lysis buffer (Qiagen). Sample collection and mRNA purification for in vivo studies were performed as follows.

Total RNA from all collected samples was purified using Qiagen RNeasy Kit following the manufacturer's protocol. Relative quantification of target mRNA and 18S ribosomal RNA gene expression was carried out with cDNA Reverse Transcription Reagents from Applied Biosystems followed by Taqman Gene Expression Assays (Applied Biosystems) using the manufacturer's protocol. The catalog numbers for each probe set were: human KIF11 (Hs00946303_ml), human AHSA1 (Hs00201602_ml), human beta-Actin (4352935E), mouse KIF11 (Mm01204215_ml), mouse AHSA1 (Mm00524718_ml), mouse beta-Actin (4352933E), and 18S (4319413E).

Comparative Microarray.

The MV4-11 suspension cells were grown in tissue culture following the manufacturers recommended conditions. Total RNA was isolated from cells using the Qiagen RNeasy Mini Kit (QIAGEN, Valencia, Calif.) and quality was assessed on the Agilent Bioanalyzer 2100 (Santa Clara, Calif.). 15 μg of total RNA was converted into cDNA and cRNA according to the manufacturer's recommendation and using manufacturer's kits (One-cycle cDNA Synthesis Kit with the addition of reagents from the Poly-A RNA Control Kit, IVT Labeling Kit, Sample Cleanup Module, Control Kit and Control Oligo B2, Affymetrix, Santa Clara, Calif.). Hybridization Mix was hybridized first to Affymetrix Human Genome U133 plus 2.0 Arrays. Staining and washing steps were performed as suggested by the manufacturer (Affymetrix, Santa Clara, Calif.). Each hybridized Affymetrix GeneChip array was scanned with a GeneChip Scanner 3000 7G (Agilent/Affymetrix). Image analysis was done with the Affymetrix GCOS software.

For the statistical analysis of the expression measurements, an in-house implementation of the RMA algorithm was used to perform the background correction, normalization and signal summarization. A multifactor ANOVA model with linear contrasts was applied to identify differentially expressed genes as a result of compound treatments and time effects using the Partek Genomic Suite (Partek Inc., St. Louis, Mo.).

The results of mRNA knockdown using the VLA-4 targeted lipid formulations of 54F, 54I, 54L, and 540 are shown in FIG. 1. As shown in FIG. 1, the solvent DMSO with no formulation was added to a suspension of MV4-11 cells as a control resulting in no knockdown of the expression of KIF11. In addition, a small molecule antagonist for inhibiting VLA-4 of (S)-2-(2-Chloro-6-methyl-benzoylamino)-3-[4-(2,6-dichloro-benzoylamino)-phenyl]-propionic acid (27):

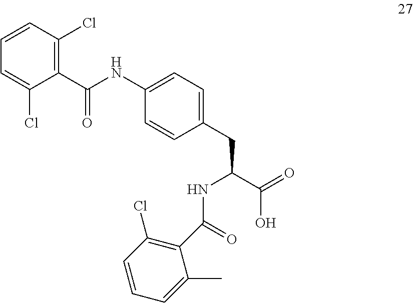 [SMILES: Cc1cccc(Cl)c1C(=O)N[C@@H](Cc1ccc(NC(=O)c2c(Cl)cccc2Cl)cc1)C(=O)O]

was also added to a suspension of MV4-11 cells with no formulation as a control resulting in no knockdown of the expression of KIF11. Formulations 54F (containing the VLA-4-targeting lipid of example 1) showed some knockdown of KIF11 expression in both the absence and presence of the above VLA-4 small molecule inhibitor (see DMSO bar vs. VLA-4 inhibitor bar which was used as a competitor for VLA-4 binding). Likewise, formulation 541 (containing the VLA-4-targeting lipid of example 3) also showed some knockdown of KIF11 expression in both the absence and presence of the above VLA-4 inhibitor. Formulation 54L (containing the VLA-4-targeting lipid of example 5) and formulation 540 (containing the VLA-4-targeting lipid of example 4) also both resulted in knockdown of KIF11 expression in both the absence and presence of the above VLA-4 inhibitor. However, for these formulations significantly more knockdown occurred in the absence of the above VLA-4 inhibitor suggesting that binding of the targeting lipid (of examples 4 and 5) to the VLA-4 receptor on the MV4-11 cells was important for increasing the delivery and knockdown ability of the siRNA targeting KIF11.

VLA-4/VCAM Binding Assay in Jurkat Cells IC₅₀.

The following adhesion assay has been reported previously and used in this invention with minor modification. See U.S. Pat. Nos. 6,229,011 and 6,380,387 both of which are incorparated herein by reference in their entirety. The functional in vitro potency of the VLA-4 (α4β1) targeting lipids (from the examples) was determined using a Jurkat cell-based assay (below) since Jurkat cells express high levels of VLA-4 on their membrane surface. Each assay was conducted in a 96-well plate with VCAM-1 used as the counter ligand for the cells (i.e., VCAM-1 was bound to the surface of the wells).

More specifically, 96-well high-binding F96 Maxisorp immuno microtiter plates (Nunc) were coated overnight with 25 ng/well of VCAM-1. On the day of the experiment, plates were blocked for 1 h with PBS buffer containing 1% nonfat dry milk to eliminate nonspecific binding. The plates were then washed with DPBS (Dulbecco's Phosphate Buffered Saline) and blotted dry. Any excess liquid was carefully aspirated from the wells.

As a control, the small molecule antagonist (27) for inhibiting VLA-4 referred to above was added to control wells in buffer containing 4% DMSO and diluted down the plate, typically in a concentration range of 1000 nM to 0.2 nM. Jurkat Clone E6-1 cells (ATCC) were labeled with 100 μg/mL 6-carboxyfluorescein diacetate, a fluorescent dye, and then activated with RPMI 1640 medium containing 0.5 mM of the divalent cation Mn²⁺ and 0.05% bovine serum albumin. It is noted that this activation is needed to achieve maximal binding for the ligand and may simulate the activation of integrins by cytokines and chemokines in vivo. The control compound (27) was determined to have an an IC₅₀ of about 12 nM (i.e. 50% of the cells did not bind to VCAM-1 on the surface of the wells since the VLA-4 receptors of the cells were presumably bound to or associated with the control compound).

To assess VLA-4 inhibition of the VLA-4 targeting lipids (from the examples), Jurkat cells (expressing high levels of VLA-4) were added to the VCAM-1-coated plates to a final concentration of 2×10⁵ cells/well in 96-well plates and allowed to incubate with the test lipids (from the examples) for 45 min at 37° C. After removing unbound cells by gently washing the wells with PBS, the fluorescence signal from the bound cells was read on a Tecan Safire2 microplate reader at 450 nm. Points were plotted and the IC₅₀s of each lipid were determined by regression analysis using the linear portion of the concentration-response curve. These results are shown below in the Table 7.

TABLE 7
IC₅₀ (nM) of VLA-4/VCAM binding in Jurkat Cells for lipids of the invention.

| Example | | IC₅₀ |
|---|---|---|
| No. | Compound Name | (nM) |
| 1 | [2,3-bis(dodecyloxy)propyl][3-[[3-[2-[2-[2-[2-[2-[2-[2-[2-[[3-[[2-[1- | 587 |
| | [[[(S)-1-carboxy-2-[4-(2,6-dichloro-benzoyl-amino)phenyl]ethyl]amino] | |
| | carbonyl]cyclo-pentyl]ethyl]amino]-3-oxopropyl]oxy]ethoxy]ethoxy] | |
| | ethoxy]ethoxy]ethoxy]ethoxy]ethoxy]ethoxy]-1-oxopropyl]amino]propyl- | |
| | dimethylaminiumtrifluoroacetate | |
| 2 | [2,3-bis(dodecyloxy)propyl][3-[[4-[[2-[2-[2-[2-[2-[2-[2-[2-[2-[2-[2-[2-[[3- | 942 |
| | [[2-[1-[[[(S)-1-carboxy-2-[4-(2,6-dichloro-benzoylamino)phenyl]ethyl] | |
| | amino]carbonyl]cyclopentyl]ethyl]amino]-3-oxopropyl]oxy]ethoxy] | |
| | ethoxy]ethoxy]ethoxy]ethoxy]ethoxy]ethoxy]ethoxy]ethoxy]ethoxy] | |
| | ethoxy]ethyl]amino]-1-4-dioxobutyl]amino]propyl]dimethylaminium- | |
| | trifluoro-acetate | |
| 3 | [[2,3-bis(hexadecyloxy)propyl][3-[[3-[2-[2-[2-[2-[2-[2-[2-[2-[[3-[[2-[1- | 1000 |
| | [[[(S)-1-carboxy-2-[4-(2,6-dichloro-benzoylamino)phenyl]ethyl]amino] | |
| | carbonyl]cyclopentyl]ethyl]amino]-3-oxopropyl]oxy]ethoxy]ethoxy] | |
| | ethoxy]ethoxy]ethoxy]ethoxy]ethoxy]ethoxy]-1-oxopropyl]amino]propyl] | |
| | dimethylaminiumtrifluoroacetate | |
| 4 | [2,3-bis(hexadecyloxy)propyl][3-[[4-[[2-2-[2-[2-[2-[2-[2-[2-[2-[2-[2-[2- | 308 |
| | [[3-[[2-[1-[[[(S)-1-carboxy-2-[4-(2,6-dichloro-benzoylamino)phenyl] | |
| | ethyl]amino]carbonyl]cyclopentyl]ethyl]amino]-3-oxopropyl]oxy]ethoxy] | |
| | ethoxy]ethoxy]ethoxy]ethoxy]ethoxy]ethoxy]ethoxy]ethoxy]ethoxy] | |
| | ethoxy]ethyl]amino]-1,4-dioxobutyl]amino]propyl]dimethylaminium- | |
| | trifluoroacetate | |
| 5 | (S)-Alpha-[[[1-[2-[[3-[2-[2-[2-[2-[2-[2-[2-[2-[2-[2-[2-[2-[[4-[[3-[[2,3- | 6 |
| | bis(hexadecyloxy)propyl]methylamino]propyl]amino]-1,4-dioxobutyl] | |
| | amino]ethoxy]ethoxy]ethoxy]ethoxy]ethoxy]ethoxy]ethoxy]ethoxy] | |
| | ethoxy]ethoxy]ethoxy]ethoxy]-1-oxoproyl]amino]ethyl]cyclopentyl] | |
| | carbonyl]amino]-4-(2,6-dichlorobenzoylamino)benzenepropanoicacid | |
| 6 | [2,3-bis(octadecyloxy)propyl][3-[[3-[2-[2-[2-[2-[2-[2-[2-[2-[[3-[[2-[1- | 133 |
| | [[[(S)-1-carboxy-2-[4-(2,6-dichloro-benzoylamino)phenyl]ethyl]amino] | |
| | carbonyl]cyclopentyl]ethyl]amino]-3-oxopropyl]oxy]ethoxy]ethoxy] | |
| | ethoxy]ethoxy]ethoxy]ethoxy]ethoxy]ethoxy]-1-oxopropyl]amino]propyl] | |
| | dimethylaminiumbromide | |
| 7 | [2,3-bis(icosanyloxy)propyl][3-[[3-[2-[2-[2-[2-[2-[2-[2-[2-[[3-[[2-[1- | 89 |
| | [[[(S)-1-carboxy-2-[4-(2,6-dichloro-benzoylamino)phenyl]ethyl]amino] | |
| | carbonyl]cyclopentyl]ethyl]amino]-3-oxopropyl]oxy]ethoxy]ethoxy] | |
| | ethoxy]ethoxy]ethoxy]ethoxy]ethoxy]ethoxy]-1-oxopropyl]amino] | |
| | propyl]dimethylaminiumbromide | |
| 8 | (S)-Alpha-[[[1-[2-[[3-[2-[2-[2-[2-[2-[2-[2-[2-[[3-[[3- | 1000 |
| | [[2,3bis(icosanyloxy)propyl]methylamino]propyl]amino]-3-oxopropyl] | |
| | oxy]ethoxy]ethoxy]ethoxy]ethoxy]ethoxy]ethoxy]ethoxy]ethoxy]-1- | |
| | oxoproyl]amino]ethyl]cyclopentyl]carbonyl]amino]-4-(2,6-dichloro- | |
| | benzoylamino)benzenepropanoicacid | |
| 9 | [2,3-bis(octadecyloxy)propyl][3-[[3-[2-[2-[2-[2-[2-[2-[2-[2-[2-[2-[2-[2-[[3- | ND |
| | [[2-[1-[[[(S)-1-carboxy-2-[4-(2,6-dichlorobenzoylamino)phenyl]ethyl] | |
| | amino]carbonyl]cyclopentyl]ethyl]amino]-3-oxopropyl]oxy]ethoxy] | |
| | ethoxy]ethoxy]ethoxy]ethoxy]ethoxy]ethoxy]ethoxy]ethoxy]ethoxy]ethoxy] | |
| | ethoxy]-1-oxopropyl]amino]propyl]dimethylaminiumtrifluoroacetate | |

1. A compound of formula I: 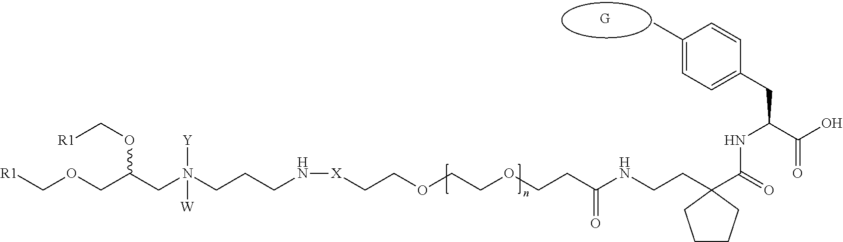
or a pharmaceutically acceptable salt or ester thereof, wherein: 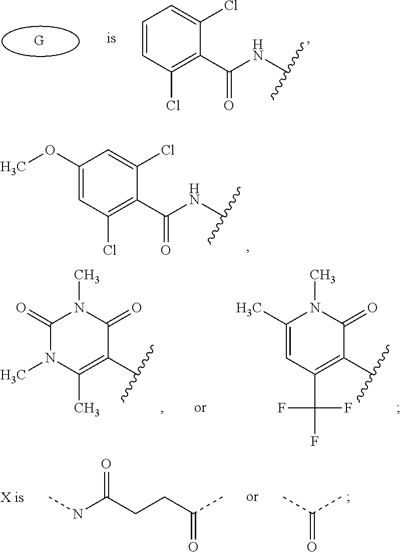
W and Y are hydrogen or methyl, wherein at least one of Y or W is methyl;
R¹ is an alkyl having 9 to 25 carbon atoms, and
n is 8 to 25.

2. A compound of claim 1 wherein:

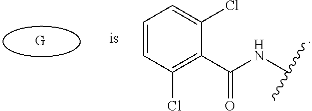 [SMILES: *NC(=O)c1c(Cl)cccc1Cl]

3. A compound of claim 1 wherein:

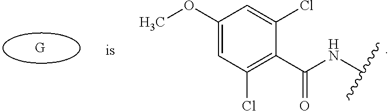 [SMILES: *NC(=O)c1c(Cl)cc(OC)cc1Cl]

4. A compound of claim 1 wherein:

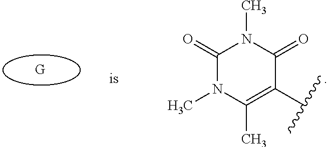 [SMILES: *c1c(C)n(C)c(=O)n(C)c1=O]

5. A compound of claim 1 wherein:

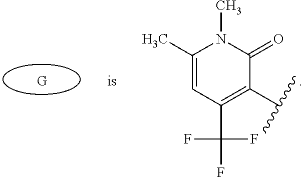 [SMILES: *c1c(C(F)(F)F)cc(C)n(C)c1=O]

6. A compound of claim 1 wherein X is:

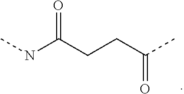

7. A compound of claim 1 wherein X is:

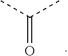

8. A compound of claim 1 wherein W is hydrogen and Y is methyl.

9. A compound of claim 1 wherein both W and Y are methyl.

10. A compound of claim 1 wherein R¹ is an alkyl having 9-20 carbon atoms.

11. A compound of claim 1 wherein R¹ is an alkyl having 16-20 carbon atoms.

12. A compound of claim 11 wherein R¹ is an alkyl having 18 carbon atoms.

13. A compound of claim 1 wherein R¹ is an alkyl having 19 carbon atoms.

14. A compound of claim 13 wherein R¹ is an alkyl having 20 carbon atoms.

15. A compound of claim 1 wherein n is 9-13.

16. A compound of claim 1 selected from the group consisting of:
[2,3-bis(dodecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate;
[2,3-bis(dodecyloxy)propyl][3-[[4-[[2-[2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1-4-dioxobutyl]amino]propyl]dimethylaminium trifluoroacetate;
[[2,3-bis(hexadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium trifluoroacetate;
[2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium trifluoroacetate;
(S)-Alpha-[[[1-[2-[[3-[2-[2-[2-[2-[2-[2-[2-[2-[2-[2-[2-[2-[[4-[[3-[[2,3-bis(hexadecyloxy)propyl]methylamino]propyl]amino]-1,4-dioxobutyl]amino]ethoxy]ethoxy]ethoxy]ethoxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino) benzenepropanoic acid;
[2,3-bis(octadecyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide;
[2,3-bis(icosanyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide;
(S)-Alpha-[[[1-[2-[[3-[2-[2-[2-[2-[2-[2-[2-[2-[[3-[[3-[[2,3-bis(icosanyloxy)propyl]methylamino]propyl]amino]-3-oxopropyl]oxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino)benzenepropanoic acid; and
[2,3-bis(octadecyloxy)propyl][3-[[3-[2-[2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethoxy]-1-oxopropyl]amino]propyl]dimethylaminium trifluoroacetate.

17. A compound of claim 1 which is:
[2,3-bis(hexadecyloxy)propyl][3-[[4-[[2-2-[2-[2-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]ethoxy]ethoxy]ethoxy]ethyl]amino]-1,4-dioxobutyl]amino]propyl]dimethylaminium trifluoroacetate

18. A compound of claim 1 which is:
(S)-Alpha-[[[1-[2-[[3-[2-[2-[2-[2-[2-[2-[2-[2-[2-[2-[2-[2-[[4-[[3-[[2,3-bis(hexadecyloxy)propyl]methylamino]propyl]amino]-1,4-dioxobutyl]amino]ethoxy]ethoxy]ethoxy]ethoxy]ethoxy]ethoxy]ethoxy]ethoxy]ethoxy]ethoxy]ethoxy]ethoxy]-1-oxoproyl]amino]ethyl]cyclopentyl]carbonyl]amino]-4-(2,6-dichlorobenzoylamino) benzenepropanoic acid.

19. A compound of claim 1 which is:
[2,3-bis(icosanyloxy)propyl][3-[[3-[2-[2-[2-[2-[2-[2-[2-[2-[[3-[[2-[1-[[[(S)-1-carboxy-2-[4-(2,6-dichloro-benzoylamino)phenyl]ethyl]amino]carbonyl]cyclopentyl]ethyl]amino]-3-oxopropyl]oxy]ethoxy]ethoxy]ethoxy]ethoxy]ethoxy]ethoxy]ethoxy]ethoxy]-1-oxopropyl]amino]propyl]dimethylaminium bromide.

20. A lipid nanoparticle composition comprising:
(1) a compound of claim 1;
(2) 2-(2,2-di((9Z,12Z)-octadeca-9,12-dienyl)-1,3-dioxolan-4-yl)-N,N-dimethyl ethanamine;
(3) a phospholipid selected from the group consisting of:
1,2-dipalmitoyl-sn-glycero-3-phosphocholine,
1,2-distearoyl-sn-glycero-3-phosphocholine, and
1,2-dioleoyl-sn-glycero-3-phosphocholine;
(4) cholesterol;
(5) hyaluronic acid; and
(6) siRNA.
